(19) United States
(12) Patent Application Publication
(10) Pub. No.: US 2021/0032693 A1
(43) Pub. Date: Feb. 4, 2021

(54) Improved Method to Analyze Nucleic Acid Contents from Multiple Biological Particles

(71) Applicant: RootPath Genomics, Inc., Boston, MA (US)

(72) Inventors: Ely Porter, Medford, MA (US); Xi Chen, Newton, MA (US)

(73) Assignee: RootPath Genomics, Inc., Boston, MA (US)

(21) Appl. No.: 16/637,454

(22) PCT Filed: Aug. 9, 2018
(86) PCT No.: PCT/US2018/045891

§ 371 Date: May 4, 2020

Related U.S. Application Data
(60) Provisional application No. 62/543,579, filed on Aug. 10, 2017.

(51) Int. Cl. C12Q 1/6869; C12Q 1/6806

(52) U.S. Cl. CPC C12Q 1/6869; C12Q 1/6806; C12Q 2521/107; C12Q 2535/122; C12Q 2522/101; C12Q 2563/159; C12Q 2563/179; C12Q 2521/325; C12Q 2563/149

(57) ABSTRACT

The application provides improved methods of analyzing biological particles and their constituents, including methods of labeling at least one target nucleic acid molecule from a biological particle with a barcoded primer.

### FIELD

This relates to a method of labeling at least one target nucleic acid molecule from a biological particle.

### BACKGROUND

Single-cell transcriptome analysis by single-cell RNA-Seq (scRNA-Seq) is a powerful approach to discover heterogeneity in gene expression profile among hundreds to hundreds of thousands of cells (Svensson et al., Nat. Methods 2017 14(4):381-387). scRNA-Seq using formalin-fixed, paraffin-embedded (FFPE) samples would be especially powerful because for retrospective studies FFPE blocks are more available, and even for prospective studies using FFPE samples makes the workflow of the study much easier due to minimal disruption of standard-of-care. Notably, FFPE samples may be a viable source of RNA with single-cell resolution, with the key observation being that intact individual nuclei can be obtained and distributed into compartments. There is representative amount of polyadenylated RNA in nuclei (Habib et al., 2016 Science 353(6302):925-928; Lacar et al., 2016 Nat. Commun., doi:10.1038/ncomms11022; Swiech et al., 2015 Nat Biotechnol 33(1):102-6; Krishnaswami 2016 Nat Protocol 11(3):499-524). Nuclei from FFPE samples have also been used for molecular analyses such as qPCR, FISH and FACS. However, there are challenges of using current scRNA-Seq methods to process FFPE sample.

A series of compositions and methods for analyzing biological particles and their constituents are described, some combination of which may result in improved scRNA-Seq methods which may allow the use of FFPE samples. Specifically, compositions and methods are provided for labeling nucleic acids from a single biological particle with barcoded primers. Some methods take advantage of the desired properties of mobile primers (e.g., high diffusion coefficient) and make using mobile primers compatible with protocols involving providing fixation reversal agent and heating of biological particles distributed in compartments. Some applications of this method include scRNA-Seq analysis of cells and nuclei from preserved samples such as frozen, FFPE-fixed, methanol-fixed, acetone-fixed, and salt-fixed (e.g., using RNAlater) samples.

### SUMMARY

In accordance with the description, in one embodiment a method of labeling at least one target nucleic acid molecule from a biological particle with a barcoded primer comprises:
a. providing a pool of at least about 100 biological particles, wherein the biological particles comprise at least one target nucleic acid molecule;
b. partitioning the pool of biological particles into compartments, wherein at least some of the compartments contain a primer delivery particle, wherein the primer delivery particle contains barcoded primers comprising at least 5 consecutive nucleotides that are complementary to at least a portion of the at least one target nucleic acid of the biological particle; and wherein the at least one barcoded primer binds to at least one target nucleic acid; and
c. inactivating barcoded primers that are not bound to a target nucleic acid.

In some embodiments, the method further comprises mobilizing the barcoded primers from the primer delivery particle.

In some embodiments, at least 50% of the compartments contain no more than one biological particle.

In some embodiments, at least 60%, 70%, 75%, 80%, 85%, 90%, 95%, 99%, or 100%of the compartments contain no more than one biological particle.

In some embodiments, at least 50% of the compartments contain no more than one primer delivery particle.

In some embodiments, at least 60%, 70%, 75%, 80%, 85%, 90%, 95%, 99%, or 100%of the compartments contain no more than one primer delivery particle.

In some embodiments, the method further comprises heating the compartments containing the biological particles to a temperature of about 60 degrees Celsius for at least about 10 minutes.

In some embodiments, the method further comprises providing one or more proteases, one or more fixation reversal agents, or any combinations thereof in the compartment.

In some embodiments, one or more fixation reversal agents comprise at least one fixation reversal catalyst.

In some embodiments, one or more fixation reversal agents comprise at least one fixation reversal enzyme.

In some embodiments, the method further comprises fixing the biological particles with one or more fixatives prior to partitioning the pool of biological particles into compartments.

In some embodiments, the method further comprises inactivating the barcoded primers that are not bound to any target nucleic acid by photo-cleaving at least one inhibitor oligonucleotide whose sequence is partially or entirely complementary to the barcoded primer.

In some embodiments, the method further comprises inactivating the barcoded primers that are not bound to any target nucleic acid by
a. providing a quencher that can bind to either the barcoded primers or the target nucleic acid under a lower temperature condition and
b. incubating the compartments at a first temperature for at least 5 minutes and then incubating the compartments at a second temperature for at least 30 seconds, wherein the second temperature is lower than the first temperature by at least 5 degrees Celsius;
c. and allowing the quencher to inactivate the barcoded primers at the lower temperature condition.

In some embodiments, the method further comprises inactivating the barcoded primers that are not bound to any target nucleic acid by
a. providing a quencher reagent that can bind to either the barcoded primers or the target nucleic acid and can be inactivated by a temperature-sensitive secondary quencher at a higher temperature condition;
b. incubating the compartments at a first temperature for at least 5 minutes and then incubating the compartments at a second temperature for at least 30 seconds, wherein the second temperature is higher than the first temperature by at least 5 degrees Celsius; and
c. allowing the quencher to inactivate the barcoded primers at the higher temperature condition.

In some embodiments, the method further comprises inactivating the barcoded primers that are not bound to any target nucleic acid with at least one inhibitor oligonucleotide whose sequence is partially or entirely complementary to the barcoded primers.

In some embodiments, the method further comprises inactivating the barcoded primers that are not bound to any target nucleic acid with at least one interfering reagent.

In some embodiments, the at least one interfering reagent comprises nucleic acid precipitants, dimethyl sulfoxide (DMSO), betaines, polyamines, urea, formamide, metal ion chelators, and combinations thereof.

In some embodiments, the inhibitor oligonucleotide or interfering reagent is in a water-in-oil emulsion.

In some embodiments, a method of labeling at least one target nucleic acid molecule from a biological particle with a barcoded primer comprises
a. providing a pool of at least 100 biological particles, wherein the biological particles comprise at least one target nucleic acid;
b. partitioning the pool of biological particles into compartments wherein at least some of compartments contain a primer delivery particle, wherein the primer delivery particle contains barcoded primers comprising at least 5 consecutive nucleotides that are complementary to at least a portion of at least one target nucleic acid of the biological particle; and wherein the at least one barcoded primer binds to at least one target nucleic acid; and
c. mobilizing the barcoded primers from the primer delivery particles before and/or after the binding of at least one barcoded primer to at least one target nucleic acid; and
d. heating the compartments accommodating the biological particles at a temperature of at least 80 degrees Celsius for at least 10 min

In some embodiments, the method further comprises at least one protease, at least one fixation reversal agent, or both.

In some embodiments, the method further comprises fixing the biological particles with one or more fixatives prior to partitioning the pool of biological particles into compartments.

In some embodiments, a method of labeling at least one target nucleic acid molecule from a biological particle with a barcoded primer comprises:
a. providing a pool of at least 100 biological particles, wherein the biological particles comprise at least one target nucleic acid;
b. partitioning the pool of biological particles into compartments, wherein at least some of the compartments contain a primer delivery particle, wherein the primer delivery particle contains barcoded primers comprising at least 5 consecutive nucleotides that are complementary to at least a portion of at least one target nucleic acid of the biological particle; and wherein the at least one barcoded primer binds to at least one target nucleic acid;
c. mobilizing the barcoded primers from the primer delivery particle before and/or after the binding of at least one barcoded primer to at least one target nucleic acid; and
d. providing a fixation reversal agent in the compartments.

In some embodiments, the method further comprises fixing the biological particles with one or more fixatives prior to partitioning the pool of biological particles into compartments.

In some embodiments, a method of labeling at least one target nucleic acid molecule from a biological particle with a barcoded primer comprises:
a. providing a pool of at least 100 biological particles, wherein the biological particles comprise at least one target nucleic acid;
b. partitioning the pool of biological particles into compartments wherein at least some of the compartments contain a primer delivery particle, wherein the primer delivery particle contains barcoded primers comprising at least 5 consecutive nucleotides that are complementary to at least a portion of at least one target nucleic acid of the biological particle, and wherein the at least one barcoded primer binds to at least one target nucleic acid; and
c. (i) mobilizing the barcoded primers from the primer delivery particle in the compartments before and/or after the binding of at least one barcoded primer to at least one target nucleic acid, (ii) after mobilizing the barcoded primers, pooling the contents of the compartments into an aqueous solution, and (iii) after pooling the contents, contacting the pooled contents in the aqueous solution with one or more nucleic acid polymerase.

In some embodiments, the nucleic acid polymerase is a RNA-dependent DNA polymerase.

In some embodiments, the RNA-dependent DNA polymerase is a reverse transcriptase.

In some embodiments, the nucleic acid polymerase is a DNA-dependent DNA polymerase.

In some embodiments, the barcoded primers are mobilized from the primer delivery particle by UV illumination, one or more reducing agents that reduce disulfide bonds, one or more enzymes that break any covalent bond between the barcoded primer and the primer delivery particle, or one or more enzymes that degrade the primer delivery particle.

In some embodiments, the median volume of the aqueous content in the compartments is 1 microLiter or less.

In some embodiments, the compartments are droplets.

In some embodiments, the biological particles are cells.

In some embodiments, at least some of the cells are prokaryotic cells.

In some embodiments, at least some of the cells are eukaryotic cells.

In some embodiments, at least some of the cells are engineered with DNA, RNA or viral vectors that encode one or more biological agents that cause RNA-mediated gene knockdown, genome editing, transcriptional alteration, or epigenetic alteration.

In some embodiments, the one or more biological agents comprise one or more of siRNA, shRNA, miRNA, zinc finger domains, transcription activator-like effector (TALE), Cas9, RNA with CRISPR origin.

In some embodiments, the target nucleic acid is RNA.

In some embodiments, the target nucleic acid is DNA.

In some embodiments, the target nucleic acid is at least part of an engineered molecule that is used to engineer or probe the biological particle.

In some embodiments, the pool of biological particles is partitioned into at least 100 compartments.

In some embodiments, at least 1% of the compartments contain a primer delivery particle.

In some embodiments, at least 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more primer delivery particles are partitioned into compartments.

In some embodiments, at least 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more biological particles are partitioned into compartments.

In some embodiments, at least some of the barcoded primers that are not bound to a target nucleic acid are inactivated in the compartments before pooling of the contents of the compartments into an aqueous solution.

In some embodiments, at least some of the barcoded primers that are not bound to a target nucleic acid are inactivated in the compartments during pooling of the contents of the compartments into an aqueous solution.

In some embodiments, at least some of the barcoded primers that are not bound to a target nucleic acid are inactivated in the compartments after pooling of the contents of the compartments into an aqueous solution.

Additional objects and advantages will be set forth in part in the description which follows, and in part will be obvious from the description, or may be learned by practice.

It is to be understood that both the foregoing general description and the following detailed description are exemplary and explanatory only and are not restrictive of the claims.

The accompanying drawings, which are incorporated in and constitute a part of this specification, illustrate several embodiments and together with the description, serve to explain the principles described herein.

### BRIEF DESCRIPTION OF THE DRAWINGS

FIG. 1 shows a container (101) having an oil phase (103) separating the compartments (here droplets) containing target nucleic acid and primer (104) and the newly added reagent in aqueous phase (102).

FIG. 2 shows droplets containing target nucleic acid and primer (201) in oil phase (203) and quenching reagent in water-in-oil droplets (202).

FIG. 3 shows quenching reagent (302) in a capsule (301), and release of the quenching reagent from the broken or permeated shell of the capsule (303).

FIG. 4 shows temporary inactivation of quenching reagent (401) using an inhibitor (402) linked to an additional recognition molecule (404), where the linker (403) can be cleaved by external trigger (405) allowing release (406) of the inhibitor and activation of the quenching reagent.

FIG. 5 shows temporary inactivation of quenching reagent (501) using an inhibitor (502) that can be converted to a non-functional form. Inactive moiety (503) does not have affinity for inhibitor (502) unless it is activated. 503 can be activated into 505 (such as through exposure to UV light) and then 505 binds to 502, inactivating the inhibitor (402) and releasing and allowing for activity of the quenching reagent (501).

FIG. 6 shows temporary inactivation of quenching reagent (601) by modifying the quenching reagent with photo-cleavable moieties (602). The quenching reagent becomes active when the photo-cleavable moieties are released.

FIG. 7 shows temporary inactivation of quenching reagent (701) by modifying the quenching reagent with complementary nucleic acid moieties (702) further comprising photo-cleavable linkers (703). When the photo-cleavable linkers are cleaved, the complementary nucleic acid moieties fall away and the quencher is activated.

FIG. 8 shows a quenching reagent that only functions at low temperature.

FIG. 9 shows a quenching reagent that only functions at high temperature.

FIG. 10 shows the workflow of Example 1.

FIG. 11 shows exemplary results of the workflow of Example 1 for the transcript GAPDH.

FIG. 12 shows the use of quenching reagents in droplets (1210) to reduce confused barcoding (1207).

### DESCRIPTION OF THE SEQUENCES

Table 1 provides a listing of certain sequences referenced herein.

TABLE 1
Description of the Sequences

| Descrip- | | SEQ ID |
|---|---|---|
| tion | Sequences | NO |
| dT₂₀ | d(TTTTTTTTTT TTTTTTTTTT) | 1 |
| dA₅₀ | d(AAAAAAAAAA AAAAAAAAAA AAAAAAAAAA | 2 |
| | AAAAAAAAAA AAAAAAAAAA) | |

### DESCRIPTION OF THE EMBODIMENTS

### I. Definitions

Barcoded primer: A barcoded primer is a primer further comprising a sequence barcode or barcodes responsible for deciphering the original location, count, or identity of the primer or the target nucleic acid. In some embodiments, the primer comprises a compartment barcode (see definition of “compartment barcode” below, referred to as a “cell barcode” in Klein et al., Cell 161:1187-1201 (2015)). In some embodiments, the primer comprises a unique molecular identifier (UMI, see Klein et al., Cell 161:1187-1201 (2015)). The barcoded primer may refer to either a forward or reverse primer or to a pair of primers (forward and reverse). In order to accomplish the barcoding, it is only necessary to bind a single barcoded primer to the target nucleic acid.

Biological particles: Biological particles are individually separable and dispersible particles of biological origin, such as cells (prokaryotic or eukaryotic), nuclei, organelles (such as mitochondria), and viruses. A biological particle is usually composed of at least 50 molecules. Other than viruses, biological particles are usually large enough that they cannot pass through 0.22-micron filter. In some embodiments, the biological particles are prepared from biological samples. For example, the biological particles can be cells prepared from fresh tissue (such as dense cell matter from tumor or neural tissues). In some embodiments, the biological particles are whole cells or nuclei prepared from frozen tissue. See, e.g., Krishnaswami. et al., Nat. Protoc. 11:499-524 (2016). In some situations, the analysis of nuclei (rather than cells) may be advantages or necessary. For example, when the cells are abnormally shaped cells (e.g. neurons) or when freezing conditions have ruptured the outer cell membrane, intact cells can be difficult to prepare, whereas intact nuclei can be prepared more readily. In some embodiments, the biological particles are nuclei prepared from FFPE tissue. In some embodiments, a biological particle is a complex of cells. The complex of cells may comprise at least two, at least three, at least four, at least five, or more cells. In some cases, the complex of cells comprises a first cell and a second cell. In some cases, the first cell is a mammalian cell. In some cases, the mammalian cell expresses a T-cell receptor or a portion thereof. In some cases, the first cell is an immune cell. In some cases, the immune cell is a T cell. In some cases, the second cell is an antigen presenting cell. In some cases, the antigen presenting cell is a dendritic cell, a macrophage, a B cell, an epithelial cell, an endothelial cell, a cancer cell or a yeast cell. In some cases, the antigen presenting cell expresses a MHC molecule on its surface.

In some cases, the MHC molecule is a class I MHC or a class II MHC. In some cases, the MHC molecule is expressed from a gene selected from HLA-A, HLA-B, HLA-C, HLA-DPA1, HLA-DPB1, HLA-DQA1, HLA-DQB1, HLA-DRA, HLA-DRB1, or any combination thereof. In some cases, the MHC molecule further comprises a peptide. In some cases, the method further comprises sequencing the barcoded target sequence.

Compartment: Compartments and partitions are used interchangeably herein and refer to microfluidic channels, wells, or droplets in which biochemical reactions (e.g., nucleic acid hybridization and primer extension) may occur. The volume of the compartment may be as large as 1 mL or as small as 1 picoLiter. In some embodiments, the median size of the compartments in one experiment is from 1 to 10 picoLiter, from 10 to 100 picoLiter, from 100 picoLiter to 1 nanoLiter, from 1 to 10 nanoLiter, from 10 to 100 nanoLiter, from 100 nanoLiter to 1 microLiter, from 1 to 10 microLiter, from 10 to 100 microLiter, or from 100 to 1000 microLiter. Examples of compartments include, but are not limited to, the single-cell GEMs of Zheng et al., Nat. Commun. 8:14049 (2017), droplets comprising single cells and gel beads as in Klein et al., Cell 161:1187-1201(2015), and the microwells of Gierahn et al., Nat. Methods 14(4):395-398 (2017). Wells in multi-well plates (e.g., 96- and 384-well plates) are also considered compartments. The volume of the aqueous content in the compartment can be smaller than or about equal to the volume of the compartment. In some embodiments, the median volume of the aqueous content in the compartments is 1 microLiter or less.

Compartment barcode: A compartment barcode is a nucleic acid sequence that is carried by primers that denote the identity of the compartment a target nucleic acid was associated with. Compartment barcode usually varies between compartments (i.e., different compartments have different compartment barcodes). At the same time, all compartment barcode sequences on all primers in one compartment usually are, or are intended to be, the same. In single cell RNA-Seq techniques such as Drop-Seq and inDrop, compartment barcodes are used as cell barcodes, in a way that all RNA transcripts from the same cell are reverse-transcribed off primers sharing the same compartment barcode. The compartment barcode is often created by clonal expansion of single template nucleic acid molecules (e.g., Church and Vigneault, US20130274117) or by split-and-pool synthesis (e.g., in inDrop and DropSeq technologies, see Klein et al., Cell 161:1187-1201 (2015) and Macosko et al., Cell 161:1202-1214 (2015), respectively). In some embodiments, a compartment barcode is a cell barcode.

Droplets: Droplets are compartments surrounded by liquid rather than solid. Droplets may be water-in-oil; water-in-oil-in-water, or water in a lipid layer (liposome). In some embodiments, the droplet can be of uniform size or heterogeneous size. In some embodiments, the median diameter of droplets used in one experiment can range from about 0.001 μm to about 1 mm. In some embodiments, the median volume of droplets used in one experiment can range from 0.01 nanoLiter to 1 microLiter.

Fixation, fixed: Fixation refers to the process of treating a biological sample (e.g., a piece of tissue or a mixture of biological particles) with one or more fixatives in order to better preserve the biological sample. Fixatives include: (a) crosslinking-based fixatives (such as formalin, formaldehyde, glutaraldehyde, paraformaldehyde, and molecules comprising two or more N-Hydroxysuccinimide esters); and (b) non-crosslinking-based fixatives. Non-crosslinking-based fixatives may comprise organic solvents (such as ethanol, methanol and acetone) or salts, or both. The salt in fixatives can be ammonium sulfate, EDTA, sodium citrate, or similar The fixative may be a hypertonic solution. In some embodiments, the hypertonic solution may be a mixture of salts where the concentration of total salt ion may be 1-5, 5-10, 10-15, 15-20, 20-30, 30-50, 50-100, 100-200, 200-300, 300-500, 500-1000, 1000-2000, or 2000 to 10000 mM. In some embodiments, the amount of ammonium sulfate in a hypertonic solution can be 5, 10, 15, 20, 30, 40, 50, 60, 65, 70, 75, 80, 90, or 100 grams in 100 mL water. In some embodiments, the hypertonic solution can be RNAlater. Biological samples that have undergone the fixation process are called fixed biological samples. Biological particles that have undergone the fixation process are called fixed biological particles.

Fixation reversal agent: A fixation reversal agent may include, but is not limited to, a fixation reversal enzyme or a fixation reversal catalyst. A fixation reversal enzyme is an enzyme that digests some content of the fixed biological sample so that the target nucleic acid is more accessible for analysis. For example, it is well known that mRNA in formalin-fixed biological samples is usually inaccessible for reverse transcription primers or enzymes due to the heavy crosslinking of the protein contents in the biological sample. Enzymes, such as proteinase K, collagenase, and hyaluronidase, can digest some protein and/or carbohydrate content of the fixed biological sample, making mRNA more accessible. Thus, proteinase K, collagenase, and hyaluronidase are examples of fixation reversal enzymes. A fixation reversal catalyst is some catalyst that aids in the reversal of the fixation. For example, this may include bifunctional transimination catalysts such as anthranilates and/or phosphoanilates that catalyze the reversal of adducts formed during formalin fixation. In some cases, the fixation reversal agent may be a reducing agent. In some cases, the reducing agent may be dithiothreitol (DTT) beta-mercaptoethanol (beta-me), and Tris (2-Carboxyethyl)-Phosphine (TCEP).

Immobile primer: Immobile primers are primers that are covalently or non-covalently bound to a primer delivery particle, or otherwise confined within a primer delivery particle. The primers confined in the gel beads in Zheng et al., Nat. Commun. 8:14049 (2017), are considered immobile primers. Immobile primers are useful to co-localize many (e.g., more than a million) copies of primers having the same compartment barcode with only 1 or a few (e.g., <10) biological particles in one compartment, so that all or a significant portion (e.g., >10%) of the target nucleic acid in the compartment is eventually copied by the primer having the identical compartment barcode.

Interfering reagent: An interfering reagent is a quenching reagent that does not specifically recognize the sequence or three-dimensional structure of the target nucleic acid or primer. For example, an interfering reagent may be nanoparticles that can non-specifically adsorb primers and target nucleic acids. When the primers and target nucleic acids are adsorbed to the surface of such nanoparticles, they can no longer freely diffuse. Thus, the interaction between the primer and the target nucleic acid will be slowed considerably. At the same time, it is possible that the nanoparticle does not cause the dissociation of pre-formed complex between the primer and the target nucleic acid. Other chemicals may also function as interfering reagent, including chemicals well known for their ability to slow nucleic acid hybridization, such as formamide and urea. Nucleic acid precipitants (e.g., mixture of salt and organic solvent), dimethyl sulfoxide (DMSO), betaines (e.g. glycine betaine), polyamines (e.g. poly-lysine or poly-ornithine, spermine, putrescine, spermidine), and metal ion chelators (e.g., ethylenediaminetetraacetic acid (EDTA) and thylene glycol-bis(β-aminoethyl ether)-N,N,N′,N′-tetraacetic acid (EGTA or egtazic acid) may also function as interfering reagent.

Mobile Primer: Primers that are not immobilized to a primer delivery particle with diameters greater than 100 nm. In Drop-Seq, the primers are attached to 30-micron-diameter bead, in which case the primer is not a mobile primer. Macosko et al., Cell 161:1202-1214 (2015). The advantages of mobile primers over immobile primers include that mobile primers are smaller and have a higher diffusion coefficient than immobile primers, thus target nucleic acids can be hybridized to mobile primers with higher efficiency.

Oligo/poly (d)A: A stretch of single-stranded nucleic acid where at least 85% of the bases are A. The stretch is usually 5- to 60-base long, and can be about 5 to 14, 15 to 20, 21 to 25, 26 to 30, 31 to 35, 36 to 40, 41 to 45, 46 to 50, 51 to 55, 56 to 60, 55 to 80, 70 to 100, or 75 to 200 bases long. Since there is no consensus cutoff between oligomer and polymer, the notation ‘oligo/poly’ is used herein. The nucleic acid may be DNA (in which case the bases are referred to as dA), RNA (in which case the bases are referred to as A), or their derivatives, such as 2′-O-methyl RNA, 2′-fluoro-RNA, LNA, PNA, morpholino, and the like. PolyA, dA₅₀, polyadenylate, and the like are all forms of oligo/poly (d)A.

Oligo/poly (d)T/U: A stretch of single-stranded nucleic acid molecule where at least 85% of the bases are T or U. The stretch is usually 5- to 60-bases long, and can be 4 to 14, 15 to 20, 21 to 25, 26 to 30, 31 to 35, 36 to 40, 41 to 45, 46 to 50, 51 to 55, or 56 to 60 bases long. Since there is no consensus cutoff between oligomer and polymer, the notation ‘oligo/poly’ is used herein. Oligo/poly (d)T/U can be used as reverse transcription primer on polyadenylated RNA. The nucleic acid may be DNA, RNA, or their derivatives such as 2′-O-methyl RNA, 2′-fluoro-RNA, LNA, PNA, morpholino, and the like. Poly dT, polyT, oligo dT, dT₂₀, and similar are all forms of oligo/poly (d)T/U.

Partition: See the Definition of Compartment.

Primer: Primers are oligonucleotides that, during an experiment or a series of experiments, become part of a molecule or a molecular complex comprising (a) the primer, and (b) a nucleic acid moiety that is either a target nucleic acid or a nucleic acid moiety whose formation is dependent on the presence or sequence of the target nucleic acid. As used herein, “primer” includes a single primer or a panel of different primers. In some embodiments, one or more of the primers may have an extendable 3′ end, may hybridize to a template nucleic acid (DNA or RNA), and/or may be extended by polymerases to copy the template nucleic acid (such as target nucleic acid). In some embodiments, one or more of the primers may be a substrate for ligation. In some embodiments, one or more of the primers may participate in a hybridization or crosslinking reaction. One or more of the primer may comprise oligo/poly (d)T/U or gene-specific sequence. The length of one or more of the primers may be from 4 to 200 nucleotides in length, in some embodiments from 80 to 160, from 120 to 140, 125 to 135, or 120 nucleotides in length. One or more of the primers may be engineered or chosen based on the features of target nucleic acid. As an example, if the target nucleic acid is polyadenylated RNA, oligo dT primer can be used as primer. The primers usually have at least 5 consecutive nucleotides that are complementary to at least a portion of the target nucleic acid. In some embodiments, one or more of the primers may contain randomly synthesized sequence. For example, random hexamer is commonly used when the sequence of target nucleic acid is unknown or diverse. In some embodiments, the primer is also associated with a unique molecular identification sequence and/or a barcode sequence.

Quenching reagent: A quenching reagent is a reagent that (a) at optimal concentration interferes with the interaction between a target nucleic acid and a primer such that the second-order rate constant for the interaction is reduced by at least 10-fold, but (b) at the above mentioned optimal concentration and under optimal experimental protocol, does not cause the dissociation of pre-formed complex between the target nucleic acid and the primer to a consequential extent, such that less than 50% of such pre-formed complex is dissociated during the experiment. In some embodiments, the quenching reagent is partial, entire, or multiple copies of the reverse complement of a barcoded primer. In some embodiments, the quenching reagent can be the synthetic molecule that mimics the partial, full, or multiple copies of the target nucleic acid. In some embodiments, the quenching reagent is latent during the association of the primer with target nucleic acid and may be activated at an optimal condition to inactivate the free primer. In some embodiments, the quenching reagent can contain a function that allows for it to be inactivated or removed. In one embodiment, the primer can be a dT₂₀ oligonucleotide with the target nucleic acid being polyadenylated RNA. In this example, synthetic dA₅₀ oligonucleotide, which (a) is multiple (i.e., 2.5) copies of the reverse complement of the barcoded primer and (b) mimics the polyA tail of the target nucleic acid, can be used as a quenching reagent. If the target nucleic acid or barcoded primer is a single-stranded nucleic acid, proteins that bind single-stranded nucleic acids such as RecA can function as quenching reagent. If polyadenylated RNA is the target nucleic acid, PolyA binding protein can function as quenching reagent.

Primer delivery particle: Beads, hydrogels, hollow particles, and the like that can host primer(s). Examples of primer delivery particles include the gel bead GEMs in Zheng et al., Nat. Commun. 8:14049 (2017), the gel beads in Klein et al., Cell 161:1187-1201 (2015), and the methacrylic polymer bead in Macosko et al., Cell 161:1202-1214 (2015). In some embodiments, the primer delivery particle may be a droplet such as a water in oil droplet or lipid microsphere that contains the primers internally in an aqueous solution. In some embodiments, the diameter of a primer delivery particle can be about from 1 micron to 1 millimeter. The primer delivery particle can also be of uniform or heterogeneous volume. The average volume of a batch of primer delivery particles used in one experiment may be from 0.5 femtoLiter to 0.5 microLiter. A primer delivery particle may also be considered ‘solid’ or describable as a soft, compressible, yet non-fluidic material such as agarose gel, polyacrylamide gel, and polydimethylsiloxane (PDMS). The primer delivery particle may host primers within, on the surface, or throughout the material comprising the particle. In some embodiments, the primer delivery particle also hosts a unique molecular identification sequence and/or a barcode sequence and these sequences can be directly linked to the primer sequence.

Target nucleic acid: A target nucleic acid is the nucleic acid selected for analysis, wherein the analysis can be any procedure that produces a human- or computer-observable signal. The analysis may comprise polymerase chain reaction (PCR), quantitative PCR (qPCR), Sanger sequencing, NextGen sequencing (using platforms such as Illumina MiSeq, Illumina HiSeq, Illumina NextSeq, Illumina NovaSeq, Ion Torrent, SOLiD, Roche 454, and the like), and the like. The analysis may yield information about the sequence or quantity of the target nucleic acid. A target nucleic acid can be DNA, RNA, or modified nucleic acid. The target nucleic acid may be the entirety or a subset of the genome or the transcriptome. The target nucleic acid may be endogenous to the biological particle it resides in (i.e., it is in the biological particle without human intervention), or be exogenous to the biological particle it resides in (i.e., it is in the biological particle due entirely or partly to human intervention). The target nucleic acid may be exogenously expressed mRNA, shRNA, non-coding RNA, or guide RNA (for the CRISPR/Cas9-based system). The target nucleic acid may contain a barcode sequence. The target nucleic acid may be a synthetic nucleic acid molecule that is conjugated to a detection probe, such as monoclonal antibody. Sometimes the original target nucleic acid one intends to analyze is converted to another molecular species or molecular complex such as a hybridization product, a primer-extension product (where the original target nucleic acid acts as the template or primer), a PCR product (where the original target nucleic acid acts as the template), a ligation product (where the original target nucleic acid acts as the splint, the 5′ ligation substrate or the 3′ ligation substrate). The newly created molecular species or molecular complexes can also be considered target nucleic acid.

### II. Components for Improved Methods of Analyzing Biological Particles and Their Constituents

The general strategy for improved methods of analyzing biological particles and their constituents involves several steps as outlined below. In some embodiments, compositions and methods are provided for labeling nucleic acids from a single biological particle with barcoded primers.

A. Preparation of Biological Particles

An aspect of the disclosure provides the means to provide biological particles (i.e., prepare biological particles in such a way as to ready them for compartmentalization). In some embodiments, the number of provided biological particles can be about 1, 2, 3, 4, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of provided biological particles can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of provided biological particles can be less than about 5, 10, 50, 100, 250, 500, 750, 1000, 1500, 2000, 2500, 5000, 7500, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of provided biological particles can be about 5-10000000, 5-5000000, 5-1000000, 10-10000, 10-5000, 10-1000, 1000-6000, 1000-5000, 1000-4000, 1000-3000, or 1000-2000.

In some embodiments, the biological particles do not need to be prepared beyond standard washing and incubation, for example, if they are cells in suspension such as peripheral blood mononuclear cells (PBMCs) and/or pre-dissociated cells. In some embodiments, the biological particles need to be prepared into suspension.

In some embodiments, the biological sample is dissociated by mechanical means. Mechanical separation can be serial passage through a constrictive device such that shearing forces pull biological particles apart. A constrictive device can be a large bore pipet tip, a Pasteur pipet, a Dounce homogenizer, or similar Mechanical separation can be achieved by passing the biological particles through the constriction a number of times. The number of passages can be about 1, 2, 3, 4, 5, 6, 8, 10, 15, 20, 25, 30, 40, 50, or more. The number of passages can be at least about 1, 2, 3, 4, 5, 6, 8, 10, 15, 20, 25, 30, 40, 50, or more. The number of passages can be less than about 1, 2, 3, 4, 5, 6, 8, 10, 15, 20, 25, 30, 40, 50, or more. The number of passages can be about 1 to 50, 1 to 10, 2 to 10, 5 to 10, 5 to 20, 5 to 30, 10 to 20, 10 to 30, 10 to 40, 15 to 20, 15 to 30, 20 to 30, 20 to 40, 20 to 50, or 30 to 50.

In some embodiments, fixation reversal agent(s) are used to facilitate the dissociation of the biological sample. A fixation reversal agent can be used to reverse the connective material fixating cells. The fixation reversal agent can be a fixation reversal agent, such as, but not limited to collagenase, hyaluronidase, trypsin, or similar The fixation reversal agent can be a combination of agents. The fixation reversal agent can be provided in the amount suggested by the manufacturer to digest a given amount of substrate for a given time and temperature. The biological particle can be treated with about 1, 2, 3, 4, 5, 10, 15, 20, 25, 30, 50, 100, 150, 200, 250, or 500 times the amount suggested for the estimated content in the sample. The biological particle can be treated with at least about 1, 2, 3, 4, 5, 10, 15, 20, 25, 30, 50, 100, 150, 200, 250, or 500 times the amount suggested for the estimated content in the sample. The biological particle can be treated with less than about 1, 2, 3, 4, 5, 10, 15, 20, 25, 30, 50, 100, 150, 200, 250, or 500 times the units suggested for the estimated content in the sample. The temperature for incubation can be about 20, 25, 30, 35, 37, 40, 45, 50, 55, 60, 65, 70, or 75° C. The temperature for incubation can be at least about 20, 25, 30, 35, 37, 40, 45, 50, 55, 60, 65, 70, or 75° C. The temperature for incubation can be less than about 20, 25, 30, 35, 37, 40, 45, 50, 55, 60, 65, 70, or 75° C. The time can be about 5, 10, 15, 20, 25, 30, 40, 45, 50, 55 minutes or 1, 1.5, 2, 2.5, 3, 4, 5, 6, 7, 8, 10, 12, 14, 16, 18, 20, or 24 hours. The time can be at least about 5, 10, 15, 20, 25, 30, 40, 45, 50, 55 minutes or 1, 1.5, 2, 2.5, 3, 4, 5, 6, 7, 8, 10, 12, 14, 16, 18, 20, or 24 hours. The time can be less than about 5, 10, 15, 20, 25, 30, 40, 45, 50, 55 minutes or 1, 1.5, 2, 2.5, 3, 4, 5, 6, 7, 8, 10, 12, 14, 16, 18, 20, or 24 hours.

In some embodiments, chemicals are used to facilitate the dissociation of the biological sample. The chemical used can be a denaturant, such as urea or guanidinium, or a chelating agent, such as EDTA. The chemical can also be a detergent, such as Triton X-100, Tween 20, Nonident P₄₀ (NP₄₀), IGEPAL CA-630, or similar The detergent may be an ionic detergent or a non-ionic detergent. The detergent may be sodium dodecyl sulfate (SDS), deoxycholate, cholate, sarkosyl, triton X-100, DDM, digitonin, tween 20, tween 80, CHAPS (3-((3-cholamidopropyl) dimethylammonio)-1-propanesulfonate).

The concentration of a chemical can be about 1, 2, 5, 10, 15, 20, 30, 50, 100, 200, 300, 500, 1000, or 2000 mM in water or buffer. The concentration of a chemical can be at least about 1, 2, 5, 10, 15, 20, 30, 50, 100, 200, 300, 500, 1000, or 2000 mM in water or buffer. The concentration of a chemical can be less than about 1, 2, 5, 10, 15, 20, 30, 50, 100, 200, 300, 500, 1000, or 2000 mM in water or buffer. The concentration of detergent can be about 0.1, 0.5, 1, 2, 3, 4, 5, 6, 8, 10, 15, 20, 25, or 30% v/v in water or buffer. The concentration of detergent can be at least about 0.1, 0.5, 1, 2, 3, 4, 5, 6, 8, 10, 15, 20, 25, or 30% v/v in water or buffer. The concentration of detergent can be less than about 0.1, 0.5, 1, 2, 3, 4, 5, 6, 8, 10, 15, 20, 25, or 30% v/v in water or buffer. The concentration of detergent can be about 0.1 to 30, 0.1 to 1, 0.1 to 5, 1 to 5, 0.5 to 1, 0.5 to 2, 0.5 to 5, 1 to 10, 5 to 10, 2 to 8, 5 to 20, 5 to 30, 10 to 20, or 10 to 30% v/v in water or buffer. The temperature of heating can be about −80, −70, −50, −20, −10, −5, −1, 20, 25, 30, 35, 37, 40, 45, 50, 55, 60, 65, 70, or 75° C. The temperature of heating can be at least about −80, −70, −50, −20, −10, −5, −1, 20, 25, 30, 35, 37, 40, 45, 50, 55, 60, 65, 70, or 75° C. or greater. The temperature of heating can be less than about −80, −70, −50, −20, −10, −5, −1, 20, 25, 30, 35, 37, 40, 45, 50, 55, 60, 65, 70, or 75° C. The temperature of heating can be about −80 to 100° C., −80 to 20° C., −20 to 0° C., 0 to 20° C., 0 to 37° C., 20 to 100° C., 20 to 75° C., 50 to 75° C., 30 to 50° C., 40 to 75° C., 75 to 100° C., or 75 to 90° C. The time of heating can be about 5, 10, 15, 20, 25, 30, 40, 45, 50, or 60 minutes. The time can be about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time of heating can be at least about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time for heating can be less than about 5 minutes, 15 minutes, 30 minutes, 45 minutes, or 1, 1.5, 2, 2.5, 3, 4, 5, 6, 7, 8, 10, 12, 14, 16, 18, 20, or 24 hours. In some embodiments, the chemical is a combination of denaturant, chelator, and/or detergent.

In some embodiments, the sample is dissociated with targeted separation. In targeted separation, a microscope or visual aid is used to select individual cells from tissue in a manual or automated fashion. An example is laser capture microdissection.

In some embodiments, the sample dissociation may be incomplete. Incomplete dissociation can be a mixed suspension of single cells and intact tissue. The mixture can be partitioned by filtering. The filter can be about a 10, 20, 30, 35, 40, 50, 70, or 100 μm nylon mesh.

In some embodiments, the sample is dissociated by a combination of dissociation methods. In some embodiments, this can be enzymatic treatment of a biological sample followed by mechanical separation of individual particles. In some embodiments, as with very difficult preserved tissue, the sample may be washed in a solvent.

In some embodiments, the dissociated sample may be enriched for a specific population or multiple populations by FACS, MACS, or similar.

B. Co-Partitioning Biological Particles and Primer Delivery Particles

The disclosure involves partitioning biological particles and primer delivery particle (which may contain immobilized primers) into compartments so that in some compartments there is only one biological particle and one primer delivery particle in a compartment. Several methods have been described enabling a single biological particle to co-partition with a single primer delivery particle in a single compartment. Svensson et al., Nat. Methods (2017). In some embodiments, at least 50%, 60%, 70%, 75%, 80%, 85%, 90%, 95%, 96%, 97%, 98%, 99%, or 100% of the compartments contain zero or only one biological particle. In some embodiments, at least 50%, 60%, 70%, 75%, 80%, 85%, 90%, 95%, 96%, 97%, 98%, 99%, or 100% of the compartments contain zero or only one primer delivery particle.

The number of partitions or compartments employed can vary depending on the application. For example, the number of partitions or compartments can be about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be less than 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partitions or compartments can be about 5-10000000, 5-5000000, 5-1000000, 10-10000, 10-5000, 10-1000, 1000-6000, 1000-5000, 1000-4000, 1000-3000, or 1000-2000.

The number of biological particles (including cells and other types of biological particles) that are partitioned into compartments can be about 1, 2, 3, 4, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of cells that are partitioned into compartments can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of cells that are partitioned into compartments can be less than 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of cells that are partitioned into compartments can be about 5-10000000, 5-5000000, 5-1000000, 10-10000, 10-5000, 10-1000, 1000-6000, 1000-5000, 1000-4000, 1000-3000, or 1000-2000.

In some embodiments, two independent types of particles can be partitioned into a single compartment. These independent particles may be biological, solid vessel, primer containing, labeling, or altogether different particles. The compartments described are relatively agnostic to the composition of the particle. In some embodiments, such as in many scRNA-Seq methods, biological particles and primer delivery particles may co-occupy a compartment.

In some embodiments, it is desired to have no more than 1 primer delivery particle in a compartment that also includes a biological particle. For example, if primer delivery particles comprise barcoded primers that contain compartment barcode, it is desirable to label all target nucleic acids from the biological particle in the compartment with one compartment barcode rather than multiple compartment barcodes (Klein et al., (2015) Cell 161: 1187; Zheng et al., (2017) Nat Commun 8: 14049; Macasco et al., (2015) Cell 161: 1202). In many methods (such as Macasco et al., (2015) Cell 161: 1202) the distribution of the number of primer delivery particles in a compartment follows Poisson distribution. For these methods, the way to minimize the occurrence of multiple primer delivery particles occupying the same compartment is to dilute the primer delivery particle, so that on average only a small fraction (e.g., 1 to 10%) of compartments contain any primer delivery particle, in which case it is very unlikely that two or more primer delivery particles co-occupy one compartment. In many of these methods, the distribution of biological particles in the compartments and the distribution of primer delivery particles in the compartments are independent. As a result, if only a small fraction of compartments include a primer delivery particle, then only a small fraction of compartments that include a biological particle also include a primer delivery particle. Nevertheless, using the methods described by Macasco et al., (2015) Cell 161:1202, Klein et al., (2015) Cell 161:1187, Zheng et al., (2017) Nat Commun 8:14049, and Gierahn et al., Nat Methods 14:395-398 (2017), one may partition the pool of biological particles into a large number (more than 100, 1000, 10,000, or 100,000) of compartments wherein at least 1% of compartment that includes a biological particle also include a primer delivery particle.

In some embodiments, the method may include providing at least 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more primer delivery particles for partitioning into compartments. For example, the method may include providing from 50000 to 200000 primer delivery particles for partitioning into compartments.

In some embodiments, the partition is an emulsion formed passively using a microfluidics device.⁷ These methods can involve squeezing, dripping, jetting, tip-streaming, tip-multi-breaking, or similar Passive microfluidic droplet generation can be modulated to control the particle number, size, and diameter by altering the competing forces of two different fluids. These forces can be capillary, viscosity, and/or inertial forces upon the mixing of two solutions.

In some embodiments, the compartments are wells in a standard microwell plate with separation aided by sorting. In some embodiments, the sorter is a fluorescence activated cell sorter (FACS). Additionally, partitioning can be coupled with automated library generation in separated microfluidics chambers, as is the case with the Fluidigm C1.

In some embodiments, the partition is a subnanoliter well and particles are sealed by a semipermeable membrane.⁸

In some embodiments, the partition is a microfluidics droplet formed by active control of a microfluidics chip. In active control, droplet generation can be manipulated via external force application, such as electric, magnetic, or centripetal forces. A popular method for controlling active manipulation of droplets in a microfluidic chip is to modify intrinsic forces by tuning fluid velocities of two mixing solutions, such as oil and water.

In some embodiments, the partition contains a primer delivery particle. In some embodiments, the primer delivery particle is a bead, hydrogel, or hollow particle. In some embodiments, the primer delivery particle can host at least one primer. In some embodiments, the primer is a barcoded primer.

In some embodiments, the primer delivery particle is a methacrylic polymer bead with immobilized primers.⁵ In some embodiments, the primer delivery particle is an acrylamide hydrogel bead with immobilized primers.^{1,3,9} In some embodiments, the primer delivery particle contains a primer as a primer for reverse transcription. In some embodiments, primer delivery particle contains a primer that can be freed from the primer delivery particle by a constitutive or inducible reagent or treatment, such as a reducing agent or UV light.

In some embodiments, the primer is barcoded primer. A barcoded primer can contain one barcode or multiple barcodes. The barcodes can be specific to the partition, specific to a given experiment, or some combination thereof. Primers can also contain a unique molecular identifier (UMI) that enables transcriptional counts post amplification during library construction. The length of the barcode or UMI can be about 1, 2, 3, 4, 5, 6, 7, 8, 10, 12, 14, 16, 18, 20, 24, 30, 35, 40, 50, or 60 nucleotides in length. The length of the barcode or UMI can be at least about 1, 2, 3, 4, 5, 6, 7, 8, 10, 12, 14, 16, 18, 20, 24, 30, 35, 40, 50, or 60 nucleotides in length. The length of the barcode or UMI can be less than about 1, 2, 3, 4, 5, 6, 7, 8, 10, 12, 14, 16, 18, 20, 24, 30, 35, 40, 50, or 60 nucleotides in length. The length of the barcode or UMI can be about 1 to 60, 1 to 40, 2 to 20, 2 to 40, 3 to 12, 2 to 8, 4 to 12, 6 to 12, 8 to 14, 10 to 20, 6 to 20, 4 to 30, or 8 to 12 nucleotides in length.

In some embodiments, many UMIs can belong to a single partition barcode, and many partition barcodes can belong to an experimental barcode. In some embodiments, the number of UMIs specific for a partition barcode can range from 1-4096. The number of UMIs per partition barcode can be about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of UMIs per partition barcode can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of UMIs per partition barcode can be less than about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of UMIs per partition barcode can be about 5-10000000, 5-5000000, 5-1000000, 10-10000, 10-5000, 10-1000, 1000-6000, 1000-5000, 1000-4000, 1000-3000, or 1000-2000.

In some embodiments, the number of partition barcodes per experimental barcode can range from 1-147,456. The number of partition barcodes per experimental barcode can be about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partition barcodes per experimental barcode can be at least about 1, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partition barcodes per experimental barcode can be less than about 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more. The number of partition barcodes per experimental barcode can be about 5-10000000, 5-5000000, 5-1000000, 10-10000, 10-5000, 10-1000, 1000-6000, 1000-5000, 1000-4000, 1000-3000, or 1000-2000.

In some embodiments, the primer is a reverse transcription primer that hybridizes to RNA template. In some embodiments, the reverse transcription primer also contains a barcode. In some embodiments, the primer is intended to hybridize to a DNA template. It should be noted that many enzymes, such as many viral reverse transcriptases, can use both DNA and RNA as template.

In some embodiments, the primer contains an element that allows the linkage between them and the primer delivery particles to be broken, allowing the primer to become a freely diffusing particle.

In some embodiments, the primer delivery particle contains an element that allows the linkage between it and the primer to be broken, allowing the primer to become a freely diffusing particle.

In some embodiments, the partition contains multiple particles. These particles can be biological, labeling, solid vessel, target, or otherwise different in nature. In some embodiments, the partition is formed in such a way as to contain a biological particle and a primer delivery particle. In some embodiments, the partition is formed in such a way as to contain a biological particle and a primer delivery particle containing a primer. In some embodiments, the biological particle is a whole cell, the primer delivery particle is a hydrogel agarose bead, the primer is photocleavable, barcoded RT primers, and the partition is a water-in-oil droplet formed by active microfluidics mixing In some embodiments, the partition may also contain a quenching reagent.

For additional examples of co-compartmentalization and co-partitioning, see U.S. Pat. No. 9,388,465 B2¹⁰ from column 15, line 16, to column 28, line 3.

C. Mobilization of Primers

In some embodiments, the primers are mobilized from the primer delivery particles. Mobilization can allow for the association of primers at rates approaching the limits of diffusion and free primers gain an entropic favorability. In the absence of mobilization, primers are reliant on the diffusion rates of the target nucleic acids to be labeled. These may be restricted due to size, steric affects, or other biological constraints and, coupled with the entropic penalty of immobilizing the primers, can lead to lower thermodynamically favorable interactions and less association of the primer with the target nucleic acids.

In some embodiments, mobilization is caused by breaking the bond connecting the primer and the primer delivery particle.

In some embodiments, the linkage between the primer and the primer delivery particle is covalent.

In some embodiments, the covalent linkage can be broken by a chemical reaction that does not otherwise affect the activity of the primer or its ability to associate with the target nucleic acid. In some embodiments, chemically labile linkage is composed of disulfides, such as cystamine or other chemically reducible linkages. Upon the addition of a reducing agent, such as beta-mercaptoethanol (BME) or dithiothreitol (DTI), the linkage is broken and the barcoded primer is mobilized, allowing it to freely associate with target nucleic acids. In some embodiments, the chemically labile linkage is a photocleavable linkage that is broken upon illumination with a specific wavelength of light. The photocleavable linkage can be a nitrobenzyl-derived linkage cleavable by illumination with about 360 nm light.¹¹ Once illuminated, barcoded primers are able to freely diffuse and label target nucleic acids. In yet another embodiment, the linkage can be thermally sensitive where elevated temperatures result in bond breakage and mobilized primers.

In some embodiments, the covalent linkage is reversible by an enzymatic reaction that does not otherwise affect the activity of the primer or its ability to associate with the target nucleic acid. In some embodiments, the enzymatically reversible linkage is a specific sequence of nucleic acid targetable by an endonuclease. Examples of sequence specific endonucleases include typeII restriction enzymes, Csy4, and RNase H. In some embodiments, the enzymatically reversible linkage is a specific amino acid sequence targeted by a protease, such as TEV. Upon exposure of the recognition sequence or moiety to the enzyme, the linkage between the primer and the primer delivery particle is broken and the primer is able to freely associate with target nucleic acids.

In some embodiments, the linkage between the primer and the primer delivery particle is non-covalent.

In some embodiments, the non-covalent linkage is a stretch of double stranded nucleic acid. The hybridized double helix can be about 5, 10, 12, 14, 16, 18, 19, 20, 22, 24, 30, 35, or 40 nucleotides in length. The hybridized double helix can be more than about 1, 2, 3, 4, 5, 10, 12, 14, 16, 18, 19, 20, 22, 24, 30, 35, or 40 nucleotides in length. The hybridized double helix can be less than about 2, 3, 4, 5, 10, 12, 14, 16, 18, 19, 20, 22, 24, 30, 35, or 40 nucleotides in length.

In some embodiments, the non-covalent bond can be broken by introduction of a competitor sequence. The competitor sequence can contain a loop or “toe-hold” sequence to facilitate strand displacement and release of the primer. In some embodiments, the hybridization can be broken by heating above the Tm of the duplex. The temperature for releasing the primer can be about 1, 2, 3, 4, 5, 6, 8, 10, 15, 20, 25, 30, 35, 40, 45, 50, 55, or 60° C. above the predicted Tm for the duplex. The temperature for releasing the primer can be at least about 1, 2, 3, 4, 5, 6, 8, 10, 15, 20, 25, 30, 35, 40, 45, 50, 55, or 60° C. above the predicted Tm for the duplex. The temperature for releasing the primer can be less than about 1, 2, 3, 4, 5, 6, 8, 10, 15, 20, 25, 30, 35, 40, 45, 50, 55, or 60° C. above the predicted Tm for the duplex. The temperature for releasing the primer can be about 1 to 60, 1 to 5, 2 to 5, 5 to 10, 5 to 20, 10 to 20, 10 to 40, 10 to 60, 15 to 30, 20 to 30, 20 to 40, 30 to 60, or 40 to 60° C. above the predicted Tm for the duplex.

In some embodiments, the duplex can also be broken by adding, removing, or altering the concentration of a chemical. In some embodiments, the chemical is a salt and the duplex is broken by reducing the available salt which aids in the formation and maintenance of the duplex. In some embodiments, the chemical is a salt and its concentration is manipulated by dilution or dialysis through a semi- or selectively-permeable membrane. In some embodiments, the chemical is a denaturant, such as urea or guanidinium. In some embodiments, the effective salt concentration can be affected by metal chelators, such as EDTA and EGTA. For example, if the solution originally contains divalent cation (such as Mg⁺⁺), the addition of EDTA can chelate Mg⁺⁺, resulting in lowered stability of duplex.

In some embodiments, the non-covalent linkage is a specific interaction between a protein and a ligand. In some embodiments, the non-covalent linkage between a protein and ligand is broken via the addition of a competitor ligand that is free in solution. In some embodiments, the protein is streptavidin, the ligand is biotin, and the reversal of the linkage can be achieved by the addition of excess biotin. In some embodiments, the protein is streptavidin, the ligand is a biotin analog with reduced affinity for streptavidin, and the linkage reversal can be achieved by the addition of biotin in an amount sufficient to outcompete the analog's interaction.

In some embodiments, a combination of treatments is used to break the linkage between a primer delivery particle and a primer. In some embodiments, the linkage is broken with a combination of heat and UV illumination. In some embodiments, the linkage is broken with enzymatic treatment and UV illumination.

In some embodiments, the linkage between a primer delivery particle and a primer is a combination of covalent and non-covalent linkages.

In some embodiments, breaking of the linkage between a primer delivery particle and a primer is achieved by breaking the primer delivery particle itself. The primer delivery particle may have a uniform structure or shelled structure containing separate constituents. In some embodiments, the primer delivery particle has a shelled structure where the primer can be confined within the particle and disruption of the shell via breaking or perforating can then release and mobilize the primers. In some embodiments, the primer delivery particle (or its shell) can be made of polymer (e.g., agarose or polyacrylamide) with reversible linkages. The reversible linkages may be moderated by either covalent or non-covalent means. A shelled primer delivery particle can be dissolved by heat, chemicals, osmotic or salt modulation, enzymes, excess ligand competition, and/or strand displacement.

In some embodiments, breaking of the linkage between a primer delivery particle and a primer is combined with or related to the release of target nucleic acids from the biological particle. In some embodiments, a combination of treatments is used to simultaneously mobilize the primer from the primer delivery particle and free the target nucleic acids from the biological particle. In some embodiments, heat is used to mobilize the primer and free the target nucleic acid. In some embodiments, heat and UV light are used to mobilize the primer and free the target nucleic acid. In some embodiments, heat, UV light, and enzymes are used to mobilize the primer and free the target nucleic acids. In some embodiments, the primer delivery particle is a hydrogel bead containing photocleavable barcoded primers as the primer, the biological particle is a whole cell or nucleus, and the target nucleic acid is polyadenylated mRNA, where UV illumination, heat, and chemicals are used to mobilize the primers and release the polyadenylated mRNA.

D. Release of Nucleic Acid Content

In some embodiments, the target nucleic acid is released from the biological particle. In some embodiments, this is performed to facilitate the association of the target nucleic acid with the primer. In some embodiments, the release of target nucleic acid from the biological particle also yields altered forms of the target nucleic acid. The altered forms can be truncated or chemically modified versions of the target nucleic acid that aid or inhibit its association with the primer.

In some embodiments, the target nucleic acid is the nucleic acid content of the biological particle. In some embodiments, the target nucleic acid may be polyadenylated mRNA.

In some embodiments, the biological particle is not preserved and target nucleic acid release can be achieved with mild treatment, such as by introducing detergent and/or mild heating. The detergent can be Triton X-100, Tween 20, NP₄₀, IGEPAL CA-630, or similar The concentration of detergent can be about 0.1, 0.5, 1, 2, 3, 4, 5, 6, 8, 10, 15, 20, 25, or 30% v/v in water or buffer. The concentration of detergent can be at least about 0.1, 0.5, 1, 2, 3, 4, 5, 6, 8, 10, 15, 20, 25, or 30% v/v in water or buffer. The concentration of detergent can be less than about 0.1, 0.5, 1, 2, 3, 4, 5, 6, 8, 10, 15, 20, 25, or 30% v/v in water or buffer. The concentration of detergent can be about 0.1 to 30, 0.1 to 1, 0.1 to 5, 1 to 5, 0.5 to 1, 0.5 to 2, 0.5 to 5, 1 to 10, 5 to 10, 2 to 8, 5 to 20, 5 to 30, 10 to 20, or 10 to 30% v/v in water or buffer. The temperature of heating can be about −80, −70, −50, −20, −10, −5, −1, 20, 25, 30, 35, 37, 40, 45, 50, 55, 60, 65, 70, or 75° C. The temperature of heating can be at least about −80, −70, −50, −20, −10, −5, −1, 20, 25, 30, 35, 37, 40, 45, 50, 55, 60, 65, 70, or 75° C. or greater. The temperature of heating can be less than about −80, −70, −50, −20, −10, −5, −1, 20, 25, 30, 35, 37, 40, 45, 50, 55, 60, 65, 70, or 75° C. The temperature of heating can be about −80 to 100° C., −80 to 20° C., −20 to 0° C., 0 to 20° C., 0 to 37° C., 20 to 100° C., 20 to 75° C., 50 to 75° C., 30 to 50° C., 40 to 75° C., 75 to 100° C., or 75 to 90° C. The time of heating can be about 5, 10, 15, 20, 25, 30, 40, 45, 50, or 60 minutes. The time can be about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time of heating can be at least about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time for heating can be less than about 5 minutes, 15 minutes, 30 minutes, 45 minutes, or 1, 1.5, 2, 2.5, 3, 4, 5, 6, 7, 8, 10, 12, 14, 16, 18, 20, or 24 hours. Once the integrity of the biological particle is disrupted, some or all of its target nucleic acid may be released.

In some embodiments, the biological particle is preserved and the target nucleic acid release can be achieved by mild treatment as discussed in the paragraph above.

In some embodiments, the biological particle is preserved and the target nucleic acid release requires processing in addition to that necessary for unpreserved samples. The additional processing required depends on the preservation methods. Some additional processing methods can be applied before the biological particles are partitioned into compartments. Some additional processing methods can be applied after the biological particles are partitioned into compartments.

In some embodiments, the biological particle is preserved by storage at low temperature. The biological particle can be frozen as a tissue sample or pellet. The biological sample can be frozen as a solution. The solution can contain a buffer, such as PBS. The solution can contain a growth media, such as EMEM, DMEM, HBSS, or similar The growth media can contain serum, such as FBS, HBS, or similar The concentration of serum in growth medium can be about 1%, 2%, 3%, 5%, 10%, 15%, 20%, 80%, 85%, 90%, or 95%. The concentration of serum in growth medium can be at least about 1%, 2%, 3%, 5%, 10%, 15%, 20%, 80%, 85%, 90%, or 95%. The concentration of serum in growth medium can be less than about 1%, 2%, 3%, 5%, 10%, 15%, 20%, 80%, 85%, 90%, or 95%.

In some embodiments, the biological particle is preserved without crosslinking in the presence of a cryoprotectant. The cryoprotectant can be DMSO or similar. The concentration of cryoprotectant can be about 1%, 2%, 3%, 5%, 10%, 15%, 20%, 80%, 85%, 90%, or 95%. The concentration of cryoprotectant can be at least about 1%, 2%, 3%, 5%, 10%, 15%, 20%, 80%, 85%, 90%, or 95%. The concentration of cryoprotectant can be less than about 1%, 2%, 3%, 5%, 10%, 15%, 20%, 80%, 85%, 90%, or 95%. The cryprotectant can be combined with a buffer such as PBS. The cryoprotectant can be combined with a growth media and the growth media can also contain serum.

In some embodiments, the biological particle is preserved or fixed by denaturation and/or precipitation. The fixative can be an alcohol or an acid. The alcohol can be methanol, ethanol, or similar The acid can be acetic acid, picric acid, or similar The fixative can be a mixture with water. he mixture can be an alcohol in water at about 1%, 2%, 5%, 10%, 20%, 30%, 40%, 50%, 60%, 70%, 75%, 80%, 85%, 90%, 95%, or 100%. The mixture can be an alcohol in water at a concentration of at least about 1%, 2%, 5%, 10%, 20%, 30%, 40%, 50%, 60%, 70%, 75%, 80%, 85%, 90%, 95%, or 100%. The mixture can be an alcohol in water at a concentration of less than about 1%, 2%, 5%, 10%, 20%, 30%, 40%, 50%, 60%, 70%, 75%, 80%, 85%, 90%, 95%, or 100%. The mixture can be an acid in water at about 1%, 2%, 5%, 10%, 20%, 30%, 40%, 50%, 60%, 70%, 75%, 80%, 85%, 90%, 95%, or 100%. The mixture can be an acid in water at least about 1%, 2%, 5%, 10%, 20%, 30%, 40%, 50%, 60%, 70%, 75%, 80%, 85%, 90%, 95%, or 100%. The mixture can be an acid in water at less than about 1%, 2%, 5%, 10%, 20%, 30%, 40%, 50%, 60%, 70%, 75%, 80%, 85%, 90%, 95%, or 100%. The fixative can be a mixture of alcohol and acid in water and/or buffer. The ratio of alcohol to acid can be about 1:1, 1:2, 1:3, 1:4, 1:5, 1:10, 1:20, 1:50. The ratio of alcohol to acid can be at least about 1:1, 1:2, 1:3, 1:4, 1:5, 1:10, 1:20, 1:50. The ratio of alcohol to acid can be less than about 1:1, 1:2, 1:3, 1:4, 1:5, 1:10, 1:20, 1:50. The ratio of acid to alcohol can be about 1:1, 1:2, 1:3, 1:4, 1:5, 1:10, 1:20, 1:50. The ratio of acid to alcohol can be at least about 1:1, 1:2, 1:3, 1:4, 1:5, 1:10, 1:20, 1:50. The ratio of acid to alcohol can be less than about 1:1, 1:2, 1:3, 1:4, 1:5, 1:10, 1:20, 1:50. The mixture can be a ratio of alcohol and acid in water or buffer at about 10%, 20%, 30%, 40%, 50%, 60%, 70%, 75%, 80%, 85%, 90%, 95%, or 100%. The mixture can be a ratio of alcohol and acid in water or buffer of at least about 10%, 20%, 30%, 40%, 50%, 60%, 70%, 75%, 80%, 85%, 90%, 95%, or 100%. The mixture can be a ratio of alcohol and acid in water or buffer at less than about 10%, 20%, 30%, 40%, 50%, 60%, 70%, 75%, 80%, 85%, 90%, 95%, or 100%.

In some embodiments, the fixative is a ketone. The ketone can be acetone or similar The ketone can be a solution with water or buffer. The concentration of ketone can be about 1%, 2%, 5%, 10%, 20%, 30%, 40%, 50%, 60%, 70%, 75%, 80%, 85%, 90%, 95%, or 100%. The concentration of ketone can be at least about 1%, 2%, 5%, 10%, 20%, 30%, 40%, 50%, 60%, 70%, 75%, 80%, 85%, 90%, 95%, or 100%. The concentration of ketone can be less than about 1%, 2%, 5%, 10%, 20%, 30%, 40%, 50%, 60%, 70%, 75%, 80%, 85%, 90%, 95%.

In some embodiments, the biological particle is preserved by a crosslinking chemical. The crosslinking chemical can be formaldehyde, glutaraldehyde, or similar The biological particle may be preserved by a solution of crosslinking chemical in water or buffer. The percentage of crosslinking chemical in solution can be about 1%, 2%, 3%, 4%, 5%, 6%, 8%, 10%, 12%, 15%, 20%, or 40%. The percentage of crosslinking chemical in solution can be at least about 1%, 2%, 3%, 4%, 5%, 6%, 8%, 10%, 12%, 15%, 20%, or 40°%. The percentage of crosslinking chemical in solution can be less than about 1%, 2%, 3%, 4%, 5%, 6%, 8%, 10%, 12%, 15%, 20%, or 40%. The percentage of crosslinking chemical in solution can be about 1 to 40%, 1 to 4%, 1 to 10%, 2 to 10%, 1 to 20%, 2 to 5%, 4 to 6%, 2 to 6%, 5 to 20%, 10 to 30%, 1 to 40%, 20 to 40% or 30 to 40%.

In some embodiments, the biological particle is preserved in a hypertonic solution. The hypertonic solution can contain high concentrations of salts. The salt can be ammonium sulfate, EDTA, sodium citrate, or similar The hypertonic solution may be a mixture of salts. The concentration of salt can be about 1-5, 5-10, 10-15, 15-20, 20-30, 30-50, 50-100, 100-200, 200-300, 300-500, 500-1000, or 1000-2000 mM. The amount of ammonium sulfate in a hypertonic solution can be about 5, 10, 15, 20, 30, 40, 50, 60, 65, 70, 75, 80, 90, or 100 grams in 100 mL water. The hypertonic solution can be RNAlater.

In some embodiments, the preserved biological particle is embedded in an immobilized medium. The immobilized medium can be a wax. The wax can be paraffin or similar The biological particle can be embedded in wax under conditions in which the wax is fluid. The wax may become fluid at elevated temperatures. Elevated temperatures can be 37, 40, 45, 50, 55, 60, 65, 70, 75, 80, 85, 90, or 95° C.

In some embodiments, the preservation conditions may contain an RNase inhibitor. The RNase inhibitor can be a protein. The protein can be produced from a recombinant source or from the biological source. The biological source can be murine serum, human placenta, or similar The RNase inhibitor can be a chemical inhibitor of RNase activity. The chemical can be DEPC, Oligo(vinylsulfonic Acid), RNAsecure, or similar The RNase inhibitor can be provided in a solution as a unit corresponding to the amount of inhibitor suggested to inhibit a given amount of RNase. The preserved biological sample can be treated with 1, 2, 3, 4, 5, 10, 15, 20, 25, 30, 50, 100, 150, 200, 250, or 500 times the units suggested for the estimated content in the sample.

In some embodiments, the biological particle is preserved by a combination of fixatives. In some embodiments, the fixative is a precipitant and/or denaturant in combination with a ketone. In some embodiments, the fixative is a precipitant and/or denaturant in combination with a crosslinking chemical. In some embodiments, the fixative is a crosslinking chemical embedded in an immobilization medium, such as FFPE samples.

In some embodiments, the preservation method can be reversed to facilitate release of nucleic acid content in the biological sample.

In some embodiments, the preservation method can be reversed by exchanging out the fixative in solution. The exchanging can be washing the sample with water, buffer, methanol, ethanol, or similar The exchanging can be a solution of alcohol in water or buffer. The concentration of alcohol can be about 5%, 10%, 15%, 20%, 25%, 30%, 40%, 50%, 60%, 70%, 75%, 80%, 85%, 90%, 95%, or 100%. The concentration of alcohol can be at least about 1%, 5%, 10%, 15%, 20%, 25%, 30%, 40%, 50%, 60%, 70%, 75%, 80%, 85%, 90%, 95%, or 100%. The concentration of alcohol can be less than about 5%, 10%, 15%, 20%, 25%, 30%, 40%, 50%, 60%, 70%, 75%, 80%, 85%, 90%, 95%, or 100%. In some embodiments, the exchanging can be washing with an organic solvent. The organic solvent can be xylene, toluene, or similar The concentration of organic solvent can be about 5%, 10%, 15%, 20%, 25%, 30%, 40%, 50%, 60%, 70%, 75%, 80%, 85%, 90%, 95%, or 100%. The concentration of organic solvent can be at least about 1%, 5%, 10%, 15%, 20%, 25%, 30%, 40%, 50%, 60%, 70%, 75%, 80%, 85%, 90%, 95%, or 100%. The concentration of organic solvent can be less than about 5%, 10%, 15%, 20%, 25%, 30%, 40%, 50%, 60%, 70%, 75%, 80%, 85%, 90%, 95%, or 100%.

In some embodiments, the preservation conditions can be reversed by modulating temperature for a given amount of time. The temperature for reversing the preservation can be about −80, −70, −50, −20, −10, −5, −1, 20, 25, 30, 35, 37, 40, 45, 50, 55, 60, 65, 70, or 75° C. The temperature for reversing the preservation can be at least about −80, −70, −50, −20, −10, −5, −1, 20, 25, 30, 35, 37, 40, 45, 50, 55, 60, 65, 70, or 75° C. or greater. The temperature for reversing the preservation can be less than about −80, −70, −50, −20, −10, −5, −1, 20, 25, 30, 35, 37, 40, 45, 50, 55, 60, 65, 70, or 75° C. The temperature for reversing the preservation can be about −80 to 100° C., −80 to 20° C., −20 to 0° C., 0 to 20° C., 0 to 37° C., 20 to 100° C., 20 to 75° C., 50 to 75° C., 30 to 50° C., 40 to 75° C., 75 to 100° C., or 75 to 90° C. The time can be about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time for reversing the preservation can be at least about 1 minute, 5 minutes, 15 minutes, 30 minutes, 45 minutes, or 1, 1.5, 2, 2.5, 3, 4, 5, 6, 7, 8, 10, 12, 14, 16, 18, 20, or 24 hours. The time for reversing the preservation can be less than about 5 minutes, 15 minutes, 30 minutes, 45 minutes, or 1, 1.5, 2, 2.5, 3, 4, 5, 6, 7, 8, 10, 12, 14, 16, 18, 20, or 24 hours.

In some embodiments, the preservation condition can be reversed by treatment with fixation reversal agents. The agent can be an enzyme such as proteinase K, hyaluronidase, glycogenase, or similar The enzyme can be provided as a unit corresponding to the amount of enzyme suggested by the manufacturer to digest a given amount of substrate at a given time and temperature. The preserved biological sample can be treated with 1, 2, 3, 4, 5, 10, 15, 20, 25, 30, 50, 100, 150, 200, 250, 500 or 1000 times the units suggested for the estimated content in the sample. The preserved biological sample can be treated with at least about 1, 2, 3, 4, 5, 10, 15, 20, 25, 30, 50, 100, 150, 200, 250, 500, or 1000 times the units suggested for the estimated content in the sample. The preserved biological sample can be treated with less than about 1, 2, 3, 4, 5, 10, 15, 20, 25, 30, 50, 100, 150, 200, 250, 500 or 1000 times the units suggested for the estimated content in the sample. The preserved biological sample can be treated with about 1 to 500, 2 to 500, 2 to 250, 2 to 150, 2 to 100, 5 to 100, 25 to 100, 50 to 100, 100 to 1000, or 500 to 1000 times the units suggested for the estimated content in the sample. The temperature for incubation can be about 20, 25, 30, 35, 37, 40, 45, 50, 55, 60, 65, 70, or 75° C. The temperature for incubation can be at least about 20, 25, 30, 35, 37, 40, 45, 50, 55, 60, 65, 70, or 75° C. or greater. The temperature for incubation can be less than about 20, 25, 30, 35, 37, 40, 45, 50, 55, 60, 65, 70, or 75° C. The temperature for incubation can be about 20 to 100° C., 20 to 75° C., 50 to 75° C., 30 to 50° C., 40 to 75° C., 75 to 100° C., or 75 to 90° C. The time of enzyme treatment can be about 5, 10, 15, 20, 25, 30, 40, 45, 50, or 60 minutes or 1, 1.5, 2, 2.5, 3, 4, 5, 6, 7, 8, 10, 12, 14, 16, 18, 20, or 24 hours. The time of enzyme treatment can be at least about 5, 10, 15, 20, 25, 30, 40, 45, 50, or 60 minutes or 1, 1.5, 2, 2.5, 3, 4, 5, 6, 7, 8, 10, 12, 14, 16, 18, 20, or 24 hours. The time of enzyme treatment can be less than about 5, 10, 15, 20, 25, 30, 40, 45, 50, or 60 minutes or 1, 1.5, 2, 2.5, 3, 4, 5, 6, 7, 8, 10, 12, 14, 16, 18, 20, or 24 hours.

In some embodiments, the fixative reversal agent is a catalyst. The catalyst can be a chemical such as bifunctional compound containing an amine and arylacid which catalyzes a transimination reaction. Examples of such bifunctional transimination chemical catalysts are the anthranilates and phosphoanilates described in Karmakar et al. Organocatalytic removal of formaldehyde adducts from RNA and DNA bases. Nat. Chem. 7: 752-758 (2015). doi:10.1038/nchem.2307, at pages 752-754¹² (incorporated by reference herein) that aid in the reversal of hemiaminal, imine, and aminal adducts formed by formaldehyde based preservation methods. The catalyst can be provided in solution at a molar concentration at a given temperature for a given amount of time in order to reverse the fixation. The concentration of catalyst can be about 1 nanoMolar, 10 nanoMolar, 100 nanoMolar, 500 nanoMolar, 1 microMolar, 5 microMolar, 10 microMolar, 20 microMolar, 50 microMolar, 100 microMolar, 250 microMolar, 500 microMolar, 1 milliMolar, 5 milliMolar, 10 milliMolar, 25 milliMolar, 50 milliMolar, 100 milliMolar, 150 milliMolar, 250 milliMolar, 500 milliMolar, 750 milliMolar, 1 molar, 1.5 molar, 2 molar, or 5 molar in concentration. The concentration of the catalyst can be about 1 nanoMolar to 5 molar, 1 nanoMolar to 100 nanoMolar, 50 nanoMolar to 500 nanoMolar, 250 nanoMolar to 1 microMolar, 500 nanoMolar to 100 microMolar, 1 microMolar to 250 microMolar, 100 microMolar to 1 milliMolar, 500 microMolar to 5 milliMolar, 1 milliMolar to 10 milliMolar, 5 milliMolar to 30 milliMolar, 10 milliMolar to 50 milliMolar, 35 milliMolar to 50 milliMolar, 35 milliMolar to 100 milliMolar, 50 milliMolar to 500 milliMolar, 250 milliMolar to 1 molar, or 500 milliMolar to 5 molar. The temperature for incubation can be about 20, 25, 30, 35, 37, 40, 45, 50, 55, 60, 65, 70, or 75° C. The temperature for incubation can be at least about 20, 25, 30, 35, 37, 40, 45, 50, 55, 60, 65, 70, or 75° C. or greater. The temperature for incubation can be less than about 20, 25, 30, 35, 37, 40, 45, 50, 55, 60, 65, 70, or 75° C. The temperature for incubation can be about 20 to 100° C., 20 to 75° C., 50 to 75° C., 30 to 50° C., 40 to 75° C., 75 to 100° C., or 75 to 90° C. The time of catalyst treatment can be about 5, 10, 15, 20, 25, 30, 40, 45, 50, or 60 minutes or 1, 1.5, 2, 2.5, 3, 4, 5, 6, 7, 8, 10, 12, 14, 16, 18, 20, or 24 hours. The time of catalyst treatment can be at least about 5, 10, 15, 20, 25, 30, 40, 45, 50, or 60 minutes or 1, 1.5, 2, 2.5, 3, 4, 5, 6, 7, 8, 10, 12, 14, 16, 18, 20, or 24 hours. The time of catalyst treatment can be less than about 5, 10, 15, 20, 25, 30, 40, 45, 50, or 60 minutes or 1, 1.5, 2, 2.5, 3, 4, 5, 6, 7, 8, 10, 12, 14, 16, 18, 20, or 24 hours.

In some embodiments, the preservation condition is reversed by a combination of treatments in order to release target nucleic acids.

In some embodiments, the treatment combination is washing and heating.

In some embodiments, the treatment combination is washing, heating, and enzymatic treatment.

In some embodiments, the treatment combination is heating and enzymatic treatment.

In some embodiments, the treatment combination is washing and enzymatic treatment.

In some embodiments, the treatment combination involves removal of an embedding medium followed by reversal of the preservation method in order to facilitate nucleic acid content release. The reversal of the preservation method can be a treatment combination.

In some embodiments, the treatment combinations for target nucleic acid release can be discontinuous or non-sequential. When the reversal of preservation is a discontinuous or non-sequential combination of treatments, a separate method can disjoint them. This disjointing method can be related to the reversal of preservation. This disjointing method can be unrelated to the reversal of preservation.

In some embodiments, the disjointing method can be a method that isolates biological particles into individual compartments. The disjointing method can be a method that co-partitions biological particles and primer delivery particles.

In some embodiments, target nucleic acid release is performed prior to the disjointing method.

In some embodiments, target nucleic acid release is performed after the disjointing method.

In some embodiments, the disjointing method is co-encapsulation of biological particles and primer delivery particles in a water-in-oil emulsion using active fluid velocity controls on a microfluidics chip.

In some embodiments, target nucleic acids are released from a preserved sample while co-partitioned with a primer delivery particle containing a primer. During this process and not bound by theory, the preservation condition is either fully or partially reversed while contained in the partition.

In some embodiments, the partition is a water-in-oil droplet containing a preserved biological sample and a solid particle containing barcoded and immobilized primers, and the preservation condition is reversed by heat and enzymatic digestion by at least one fixation reversal enzyme.

In some embodiments, the mobilization of primer is carried out before the release of target nucleic acids.

In some embodiments, the mobilization of primer is carried out after the release of target nucleic acids.

In some embodiments, the mobilization of primer and the release of target nucleic acids are carried out simultaneously. For example, if the integrity of the primer delivery particle is susceptible to protease treatment or heat, then providing protease in the compartments or heating the compartments will trigger both mobilization of label particle and the release of target nucleic acid from the biological particle.

E. Binding of the Primer to the Target Nucleic Acid

The barcoded primer, in order to label the target nucleic acid, binds to the target nucleic acid while the barcoded primer and the target nucleic acid are within the compartment.

The barcoded primer may be mobilized from the primer delivery particle before, after, or during binding with the target nucleic acid or a combination thereof (for a plurality of barcoded primers and target nucleic acids in either a compartment or a biological sample).

The conditions within the compartment may allow for binding of the primer to the target nucleic acid.

In order to achieve barcoding of the target nucleic acid, it is only necessary to bind a single barcoded primer to an individual target nucleic acid. Thus, the barcoded primer may refer to either a forward or reverse primer. In some embodiments, both forward and reverse primers may be used and the barcoded primer may be a pair of primers (forward and reverse). Forward and reverse primers may have the same barcode or they may have a pair of barcodes associated with each other.

In some embodiments, a single bar code sequence may be associated with two different primers when the goal is to label, and subsequently identify, two nucleic acid sequences that are in the same compartment and/or associated with each other. For example, to sequence heavy and light chains on immune cells, a primer to the heavy chain and a primer to the light chain may be used, each associated with the same barcode for each compartment. The primer for the light chain and the primer for the heavy chain may also have a pair of barcodes associated with each other.

F. Making Enzymatic Primer Extension Compatible with High-Temperature Treatment

One possible challenge of using mobile primers as opposed to immobile primers in compartmentalization-based (e.g., droplet-based) analysis of biological particles (e.g., single cells RNA transcriptome analysis, see^{1,3,5}) is that once the content from multiple compartments are pooled together (e.g., when emulsion is broken) a free primer may bind to a target nucleic acid (e.g., mRNA molecule) that did not co-reside in the same compartment as the primer delivery particle (e.g., the hydrogel bead) from which the mobile primer is mobilized. If the primer contains a compartment barcode, this binding may lead to misassignment of the target nucleic acid. In other words, nucleic acid targets from different biological particles which resided in different compartments may be erroneously assigned the same compartment barcode. This process is sometimes called “confused barcoding” or “confounded barcoding.” In some methods where mobile primers are used in the compartments (e.g., Klein et al.¹), this problem is solved by completion of the reverse transcription reaction and inactivating the reverse transcriptase within the compartments before pooling the contents from different compartments (i.e., breaking the emulsion). This way, after breaking the emulsion, even if a primer binds to a RNA transcript from a cell that resided in a different compartment, it does not undergo reverse transcription or label the RNA transcript with its compartment barcode. However, finishing the reverse transcription within the compartments requires providing reverse transcriptase and necessary cofactors (e.g., Mg⁺⁺) in the compartments. This limits the treatment that can be applied to the content of the compartments. For example, to break the crosslinking induced during some fixation methods (e.g., formaldehyde-based fixation), a protease (e.g., protease K) is often necessary. However, including protease K in the compartments can cause the degradation of the reverse transcriptase if the reverse transcriptase is also provided in the compartments Similarly, breaking the crosslinking induced during some fixation methods (e.g., formaldehyde-based fixation) may require heating at greater than 80° C., which may inactivate most types of reverse transcriptase (with notable exceptions such as RTX¹³) and may compromise the integrity of RNA in the presence of Mg⁺⁺.

In some embodiments, a highly thermostable reverse transcriptase (such as RTX) can be used in order to withstand the high temperature required to sufficiently reverse the crosslinking Or the temperature used to reverse the crosslinking may be kept at the temperature (e.g. about 60 degrees Celsius) that does not inactivate some commercially available reverse transcriptases (such as SuperScript IV). To facilitate experiments comprising heat treatment of the compartments, in some embodiments the cofactors, which may cause RNA degradation at high temperature, are temporarily shielded by a conditionally inactivated chelator. In some embodiments, the conditionally inactivated chelator is a photo-cleavable metal chelator. A number of examples of such chelators, as well as strategies to synthesize such chelators have been reported (see U.S. Pat. No. 5,709,848, also see M. A. McKinley, “Photochemical Release of Metal Ions: A Modified Caging Design of a Photocleavable Chelator for the Light Directed Release of Metal Ions”, University of Georgia, 2013). When such chelators are used, high temperature can be used to facilitate RNA release, and then the chelators can be inactivated (e.g., photo-cleaved) to release the cofactors that facilitate reverse transcription.

G. Inactivation of Free Primers

In some embodiments, it is desirable to be able to use mobile primer to assign compartment barcode to target nucleic acids without requiring that reverse transcription or primer extension are completed before pooling the contents of different compartments (e.g., breaking the emulsion).

There are many methods to achieve this goal. In some embodiments, the goal is achieved by inactivating (a) the primers that are not bound to target nucleic acid (i.e., free primer), or (b) target nucleic acids that are not bound to the primer (i.e., free target nucleic acid), or (c) both, before or during pooling contents from different compartments (i.e., before or during breaking the emulsion in the case where the compartments are droplets). In some embodiments, once inactivated, the primer carrying a compartment barcode can no longer assign such barcode to other target nucleic acids. In some embodiments, one can use (a) reagents that bind or degrade target nucleic acid, rendering such target nucleic acid unable to bind primer, (b) reagents that bind or degrade the primer, rendering such primer unable to bind target nucleic acid, and/or (c) reagents that hinder nucleic acid hybridization. These reagents are collectively referred to as “quenching reagents.”

Types of quenching reagents. In some embodiments, the quenching reagent is a protein or a nucleic acid molecule. In some embodiments, if the free target is polyadenylated RNA and the primer comprises oligo/poly (d)T/U, then poly A binding protein or oligonucleotides comprising oligo/poly (d)T/U can be used to inactivate the target nucleic acid. The quenching reagent can also be oligonucleotides that comprise sequence complementary to that of the primer, and can bind the primer when provided conditions (e.g., certain salt concentration and temperature, which can be optimized using standard method) to do so.

In some embodiments, the quenching reagent is a single-strand specific exonuclease, such as E. coli Exonuclease I. In some embodiments, the quenching reagent is an oligonucleotide comprising oligo/poly (d)A, in which case the quenching reagent can bind free primer if the free primer comprises oligo/poly (d)T/U.

In some embodiments, the quenching reagent is an interfering reagent. In some embodiments, the interfering reagent that hinders nucleic acid hybridization is a metal-ion chelator such as EDTA and EGTA. In some embodiments, the interfering reagent that hinders nucleic acid hybridization are one or more denaturants, such as formamide and urea. Such reagent can be prepared according to Simard et al.¹⁴ In some embodiments, the interfering reagent comprises components that cause precipitation of target nucleic acid or primer. In some embodiments, the components that cause precipitation of target nucleic acid or primer comprise ions such as K⁺, Na⁺, Li⁺, NH₄⁺, Ac− (acetate), Cl−, or SO₄²−. In some embodiments, the components that cause precipitation of target nucleic acid or primer comprise organic solvents such as ethanol, isopropanol, butanol and acetone. The components that cause precipitation of target nucleic acid or primer can be prepared following “UNIT 2.1A Purification and Concentration of DNA from Aqueous Solutions” by David Moore and Dennis Dowhan.¹⁵ In some embodiments, the components that cause precipitation of target nucleic acid or primer are added in a way that molecules or molecular complexes above, below or within a specific size range are preferentially precipitated. In some embodiments, the interfering reagent is nanoparticles that can non-specifically adsorb primers and target nucleic acids. When the primers and target nucleic acids are adsorbed to the surface of such nanoparticles, they can no longer freely diffuse. Thus, the interaction between the primer and the target nucleic acid will be slowed considerably. At the same time, it is possible that the nanoparticles do not cause the dissociation of pre-formed complex between the primer and the target nucleic acid.

Providing quenching reagent during the pooling of contents from compartments. In some embodiments, the quenching reagent can be provided during the pooling of the contents from compartments. The quenching reagent can be of aqueous nature (i.e., as opposed to organic/hydrophobic). In some embodiments where the compartments are created on a solid support (e.g., Gierahn et al., Nat Methods 14: 395-398 (2017)), there is no phase barrier (e.g., oil) that separates the content of the compartments and newly added aqueous quenching reagent, although a semi-permeable membrane may be used to seal the compartments. In this situation, the aqueous quenching reagent can be simply added to the compartments and the quenching reagent can contact the content of the compartments.

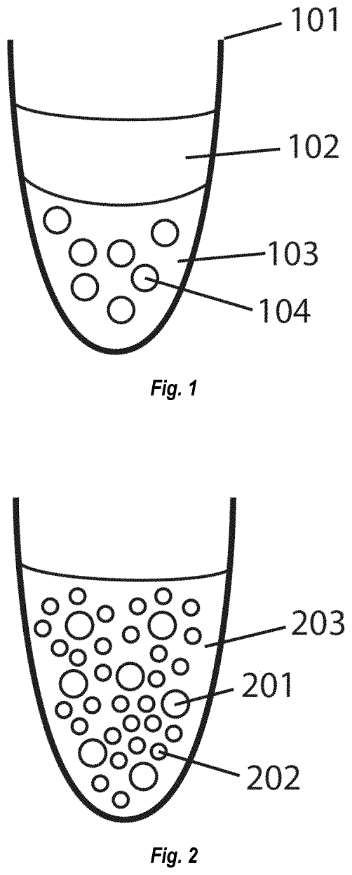

In some embodiments, there is a phase barrier between the contents of the compartments and the quenching reagent. For example, when water-in-oil droplets are used as compartments, the aqueous content of the compartment (FIG. 1, 104) is surrounded by oil (FIG. 1, 103). When the aqueous quenching reagent (FIG. 1, 102) is added to the container (FIG. 1, 101) that contains the droplets, the quenching reagent cannot contact the content of the compartment due to the presence of the oil phase (FIG. 1, 103). In some embodiments, such contact can be allowed by adding reagents that break the emulsion. Examples of emulsion-breaking reagents include ether and 20% (vol/vol) 1H,1H,2H,2H-Perfluorooctanol in HFE-7500 oil [3M Novec 7500 Engineered Fluid (HFE-7500 oil, 3 M; Novec, cat. no. Novec 7500)]. Examples of breaking emulsion have been described in the literature. Klein et al., Cell 161:1187-1201 (2015); Macosko et al., Cell 161:1202-1214 (2015); Spencer et al., epicPCR (Emulsion, Paired Isolation, and Concatenation PCR), Protoc. Exch. (2015); and Villani et al., Science 356:6335 (2017). The emulsion can also be broken by heating. The optimal temperature and incubation time can be determined by observing the speed at which emulsion breaks as a function of temperature and incubation time. In some embodiments, physical agitation (e.g., vortexing) can be applied to accelerate the contact between contents of the droplets and the quenching reagent.

In some embodiments, the quenching reagent can be formulated in the form of water-in-oil emulsions, in which the water droplets comprise the quenching reagent. Such emulsions can be made by a variety of methods such as agitation and via microfluidic devices. For example, the procedures described by Tawfik and Griffiths, Nat. Biotechnol. 16:652-656 (1998), by Klein et al., Cell 161:1187-1201 (2015) (see FIG. 2A of Klein et al.), and others (Macosko et al., Cell 161:1202-1214 (2015); Villani et al., Science 356:6335 (2017)) can be used to create emulsions, sometimes with minor modifications. In many embodiments, the size distribution of the droplet can be adjusted by the frequency or amplitude of the agitation, or by controlling the flow rate of the water or oil channels in the microfluidic device, or by controlling the dimension of the microfluidic channels in the microfluidic device. The droplets are compartments. The median volume of the droplets can be at maximum 10 microLiter. In some embodiments, the median volume of the droplets can be about 0.1 picoLiter to 1 nanoLiter, 1 nanoLiter to 1 microLiter, 1 microLiter to 10 microLiter, 0.1 to 1 picoLiter, 1 to 10 picoLiter, 10 to 100 picoLiter, 100 to 1000 picoLiter, 1 to 10 nanoLiter, 10 to 100 nanoLiter, 100 to 1000 nanoLiter, or 1 to 10 microLiter. The water-in-oil emulsion where the aqueous droplets comprise the quenching reagent can be mixed with the water-in-oil emulsion where the aqueous droplets comprise the labeling primer and target nucleic acid, after which the reagent that breaks the emulsion can be added. Formulating the quenching reagent in water-in-oil emulsions can promote that the droplets comprising the primer and target nucleic acid (FIG. 2, 201) are sufficiently surrounded by droplet comprising the quenching reagent (FIG. 2, 202). This can increase the chance that, during the breaking of emulsion, the free mobile primer is inactivated by the quenching reagent before it contacts the target nucleic acids that did not co-reside in the compartment with the free mobile primer. FIG. 2, 203 shows the oil phase. In some embodiments, the buoyancy of the droplet containing the quenching reagent can be different from that of the droplet containing the primer and target nucleic acid. In this embodiment one population of droplets may settle down and the other population of droplets may float up. This may result in inefficient mixing of the two droplets and could result in cross-contamination of compartments and their contents. Suspension reagents can then be added to the droplets containing the quenching reagent to promote the droplets staying in suspension and reduce settling. In some embodiments, the suspension reagent can be iodixanol, sucrose, glycerol, or similar In some embodiments, the concentration of suspension reagent can be about 0.5, 1, 2, 3, 4, 5, 7, 10, 12, 15, 17, 20, 22, 25, 30, 35, 40, 45, 50, 60, 70, 80, 90, or 100%. In some embodiments, the concentration of the suspension reagent can be less than about 0.5, 1, 2, 3, 4, 5, 7, 10, 12, 15, 17, 20, 22, 25, 30, 35, 40, 45, 50, 60, 70, 80, 90, or 100%. In some embodiments, the concentration of the suspension reagent can be more than about 0.5, 1, 2, 3, 4, 5, 7, 10, 12, 15, 17, 20, 22, 25, 30, 35, 40, 45, 50, 60, 70, 80, 90, or 100%. In some embodiments, the concentration range of the suspension reagent can be about 0.5-100%, 0-1%, 0.5-2%, 1-5%, 2-10%, 5-15%, 10-20%, 15-30%, 20-40%, 30-60%, 50-75%, or 60-100%. The choice and optimal concentration of suspension reagent can be determined empirically with straightforward assays. For example, the droplets comprising primer and biological particles can be labeled with one fluorescent dye, and the droplets comprising the quenching reagent can be labeled with a different fluorescent dye. The mixed emulsion can be imaged with con-focal fluorescent microscope with z-scanning to observe the density of two populations of the droplets as a function of height. The formulation of the droplets containing the quenching reagent that results in stable, height-independent relative density is preferred.

Providing quenching reagent in the compartments. In some embodiments, the quenching reagent is provided in the compartments that contain target nucleic acid and primer. In some embodiments, the quenching reagent can be engineered in the way that it is inactive for a period of time, during which the target nucleic acid can contact the primer, and then activated to inactivate the target nucleic acid or the primer, or otherwise prevent the target nucleic acid and primer from associating. This feature is referred to as the “delayed release of quenching reagent.” The release of quenching reagent may be caused by a reagent in a compartment or by external stimuli (stimuli that can be delivered without introducing material to the compartments, e.g., electro field, magnetic field, electromagnetic wave, acoustic wave, light, microwave, etc., or combination thereof). When the release of quenching reagent is caused by a reagent in a compartment, and when the compartment is a droplet, the concentration of the reagent can be tuned so that the quenching reagent is not released immediately (e.g., within seconds), but released over a long period of time (e.g., minutes to hours) to allow the primer to bind the target nucleic acid.

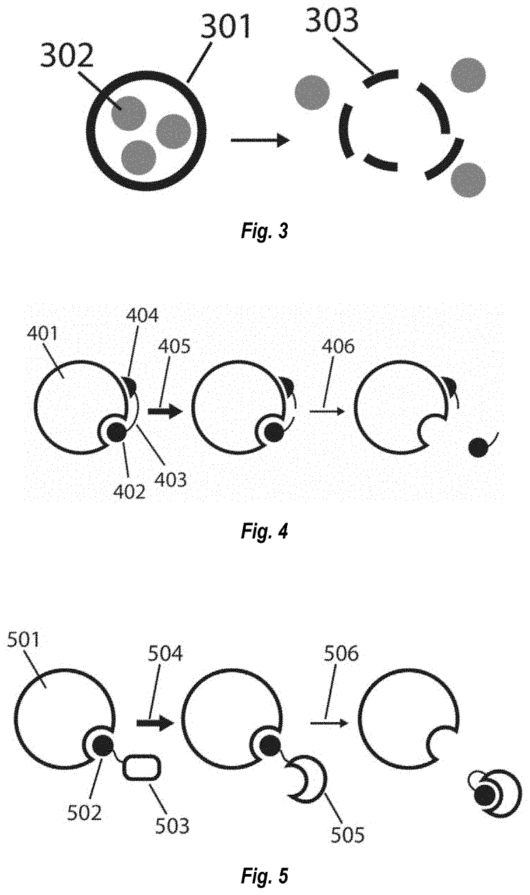

In some embodiments, the delayed release of quenching reagent is realized by encapsulating the quenching reagent in a capsule (FIG. 3, 301) whose size is smaller than the compartment and can be included in the compartments, where the shell of the capsule may be broken down or permeabilized by a reagent in the compartment or by external stimuli. FIG. 3 shows a diagram of this method. Several methods to construct and break/permeabilize such capsules have been reported (e.g., U.S. Pat. No. 9,388,465). The method taught by U.S. Pat. No. 9,388,465 can also be modified. For example, the material for the shell can comprise one or more photo-cleavable linkers, so that the shell can be broken or permeabilized using UV treatment. The shell can also comprise protein or peptides so that the shell can be broken by protease.

In some embodiments, the delayed release of quenching reagent is realized by providing a conditional inhibitor of the quenching reagent. For example, if the quenching reagent is a protein (FIG. 4, 401), one may identify a low-affinity inhibitor (FIG. 4, 402) of the quenching reagent and link the low-affinity inhibitor to an additional recognition molecule (FIG. 4, 404) that binds but does not inhibit the quenching reagent. The low-affinity inhibitor and the additional recognition molecule can be linked (FIG. 4, 403) to form the conditional inhibitor of the quenching reagent. To render the quenching reagent conditional, the linker (FIG. 4, 403) can be made cleavable by a reagent in the compartment or by external stimuli (e.g., UV light) (FIG. 4, 405), allowing for release (FIG. 4, 406) of the low-affinity inhibitor. In some embodiments, the linker comprises disulfide bond which can be cleaved by thiol in the compartment. In some embodiments, the linker comprises at least one photo-cleavable moiety.

Inhibitors that bind and inhibit the quenching reagent with desired affinity can be identified by routine methods such as high-throughput screening and medicinal chemistry-style chemical modifications. The additional recognition molecule can be a monoclonal antibody, a fragment of a monoclonal antibody, an aptamer, or the like, all of which can be generated using standard methods. The linker may also comprise flexible linkers such as ethylene glycol units. The inhibitor is considered low-affinity if it does not inhibit more than 20% of the quenching reagent if used alone at certain concentration, but inhibits more than 80% of the quenching reagent when it is linked to the additional recognition molecule and used at the same concentration. Without being bound by theory, the following example shows how such a conditional inhibitor may operate. For example, if an inhibitor has a K_{d} value of 1 microMolar, the concentration of the quenching reagent is 0.1 nanoMolar and the concentration of the inhibitor is 10 nanoMolar. Then when the inhibitor is used alone only roughly [10 nanoMolar/(1 microMolar+10 nanoMolar)=˜] 1% of the quenching reagent is expected to be bound by the inhibitor. In contrast, if the inhibitor is linked to an aptamer (i.e., the additional recognition molecule) that binds (but does not inhibit) the quenching reagent with 1 nanoMolar affinity, when the linked inhibitor is used at 10 nanoMolar (same concentration as before), [10 nanoMolar/(1 nanoMolar+10 nanoMolar)=]˜90% of the quenching reagent is expected to be bound by the aptamer. For the quenching reagent that is bound by the aptamer, if the linker and the site for aptamer binding on the quenching reagent dictates that the effective local concentration of the inhibitor is 100 microMolar, then [100 microMolar/(1 microMolar+100 microMolar)=]˜99% of the quenching reagent that is bound by the aptamer is also bound by the inhibitor. Effectively, [90%*99*=]˜89% of the quenching reagent is bound by the inhibitor when the inhibitor is linked to the additional recognition molecule and is used at the same concentration (10 nanoMolar). In some embodiments, the affinity of the low-affinity inhibitor to the quenching reagent is 1 nanoMolar to 100 microMolar, 1 nanoMolar to 10 nanoMolar, 10 nanoMolar to 100 nanoMolar, 100 nanoMolar to 1 microMolar, 1 microMolar to 10 microMolar, or 10 microMolar to 100 microMolar. In some embodiments, the affinity of the low-affinity inhibitor to the quenching reagent is about 1 nanoMolar to 100 microMolar, 1 nanoMolar to 10 nanoMolar, 10 nanoMolar to 100 nanoMolar, 100 nanoMolar to 1 microMolar, 1 microMolar to 10 microMolar, or 10 microMolar to 100 microMolar.

In some embodiments, the conditional inhibitor is created by linking an inhibitor (FIG. 5, 502) of the quenching reagent (FIG. 5, 501) and an additional moiety (FIG. 5, 503) that can be converted by a reagent present in the compartment or external stimuli (FIG. 5, 504) from a form (FIG. 5, 503) that does not bind the inhibitor to a form that binds the inhibitor (FIG. 5, 505). For example, the inhibitor can be an aptamer and the additional moiety can be nucleic acid with sequence complementary to the aptamer and further comprise photo-responsive functional groups. Upon illumination of light of certain wavelength, the photo-responsive functional groups are cleaved or altered so that the additional moiety can bind and inactivate the aptamer, rendering the inhibitor inactive (FIG. 5, 506). An example of this strategy is provided by Kim et al., Proc. Natl. Acad. Sci. 106:6489-6494 (2009).

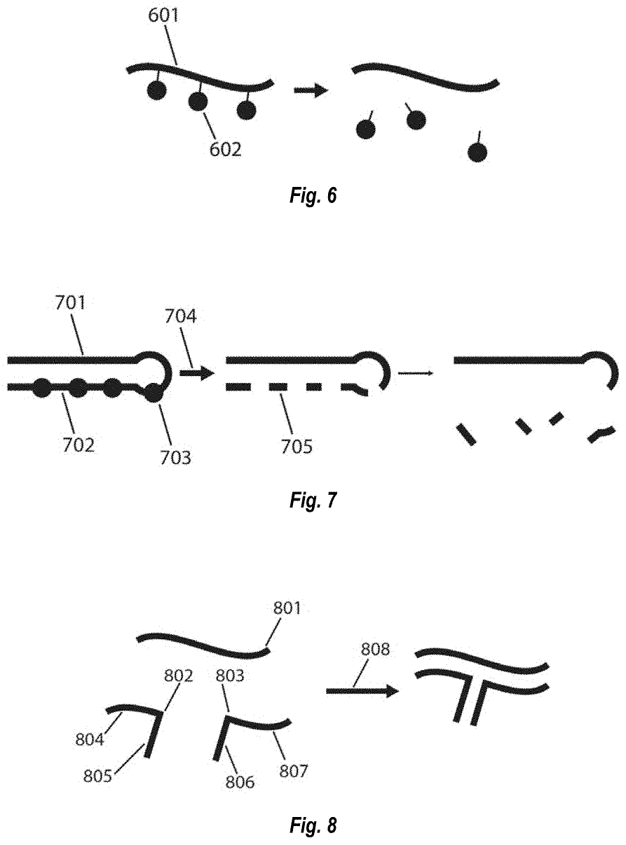

In some embodiments, the quenching reagent is modified by photo-responsive moieties. For example, if the quenching reagent is an oligonucleotide (FIG. 6, 601), it can be modified with photo-cleavable groups (FIG. 6, 602) in a way that (a) before the photo-cleavable groups are photo-cleaved, the oligonucleotide cannot bind its target (i.e., the target nucleic acid or the primer), and (b) after the photo-cleavable groups are photo-cleaved, the oligonucleotide can bind its target. An example of the strategy is described in Connelly et al., Mol. Biosyst. 8:2987 (2012).

In some embodiments, if the quenching reagent is an oligonucleotide (referred to as the first oligonucleotide, FIG. 7, 701), it can be covalently or non-covalently linked to a second oligonucleotide (FIG. 7, 702) that can hybridize with the first oligonucleotide and comprise photo-cleavable moieties (FIG. 7, 703). After light (e.g., UV) exposure (FIG. 7, 704), the photo-cleavable moieties can be cleaved, leaving short segments of the second oligonucleotide (FIG. 7, 705) which can dissociate spontaneously from the first oligonucleotide, allowing the first oligonucleotide to function as the quenching reagent. In some embodiments, the median length of the fragments is less than about 1, 2, 3, 4, 5, 6, 7, 8, 9, or 10 nucleotide(s).

In some embodiments, the quenching reagent comprises at least 2 oligonucleotides (FIG. 8, 802 and 803) that stably bind (FIG. 8, 808) a target (FIG. 8, 801), wherein (a) the first oligonucleotide (FIG. 8, 802) comprises a first domain (FIG. 8, 804) and a second domain (FIG. 8, 805), (b) the second oligonucleotide (FIG. 8, 803) comprises a first domain (FIG. 8, 806) and a second domain (FIG. 8, 807), (c) the first domain of the first oligonucleotide is complementary to a target (in this embodiment a target nucleic acid or primer, see FIG. 8, 801) and the second domain of the first oligonucleotide is complementary for the first domain of the second oligonucleotide, (d) the second domain of the second oligonucleotide is complementary to the target, and (e) the first domain of the first oligonucleotide alone and the second domain of the second oligonucleotide alone cannot stably bind the target at the condition in the experiment at which the target nucleic acid and the primer are intended to bind each other.

In some embodiments, the quenching reagent is functional only when the temperature is low, i.e., when the duplex between the second domain of the first oligonucleotide and the first domain of the second oligonucleotide can form in the presence of the target. In this embodiment, the delayed release of quenching reagent is realized by first allowing the hybridization between the target nucleic acid and primer to happen at a high temperature at which the complex comprising the target, the first oligonucleotide and the second oligonucleotide is unstable, then lowering the temperature to the temperature at which the complex comprising the target, the first oligonucleotide and the second oligonucleotide is stable. The length of the domains and the temperatures can be determined using standard assays.

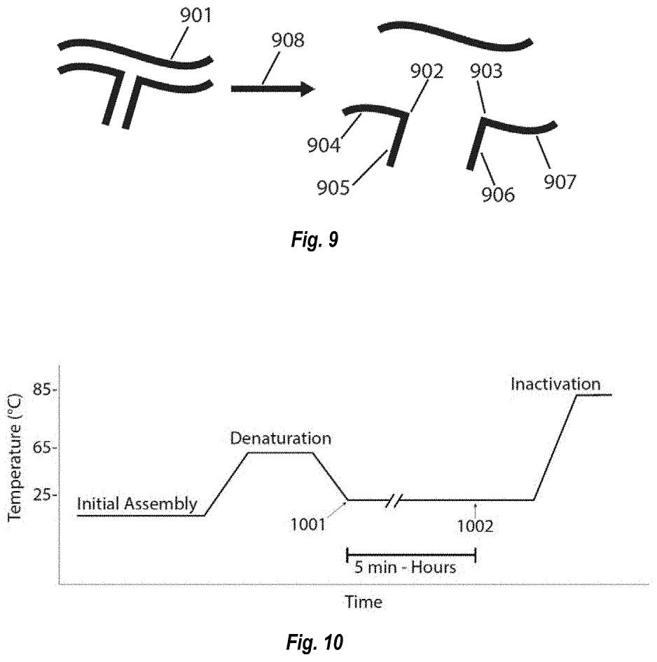

In some embodiments, a temperature-controlled inhibitor complex can be used to realize delayed release of the quenching reagent if the quenching reagent is an oligonucleotide. In some embodiments, the temperature-controlled inhibitor complex comprises at least two oligonucleotides (FIG. 9, 902 and 903), wherein (a) the first oligonucleotide (FIG. 9, 902) comprises a first domain (FIG. 9, 904) and a second domain (FIG. 9, 905), (b) the second oligonucleotide (FIG. 9, 903) comprises a first domain (FIG. 9, 906) and a second domain (FIG. 9, 907), (c) the first domain of the first oligonucleotide is complementary to the quenching reagent (FIG. 9, 901) and the second domain of the first oligonucleotide is complementary to the first domain of the second oligonucleotide, (d) the second domain of the second oligonucleotide is complementary with the quenching reagent, and (e) the first domain of the first oligonucleotide alone and the second domain of the second oligonucleotide alone cannot stably bind the quenching reagent at the condition in the experiment at which the target nucleic acid and the primer are intended to bind each other.

In some embodiments, the temperature-controlled inhibitor complex is functional only when the temperature is low, i.e., when the duplex between the second domain of the first oligonucleotide and the first domain of the second oligonucleotide can form in the presence of the quenching reagent. In this embodiment, the delayed release of quenching reagent is realized by first allowing the hybridization between the target nucleic acid and the primer to happen at a low temperature at which the complex comprising the quenching reagent, the first oligonucleotide and the second oligonucleotide is stable, then raising the temperature to the temperature at which the complex comprising the quenching reagent, the first oligonucleotide and the second oligonucleotide is unstable (FIG. 9, 908), releasing the quench strand. The length of the domains and the temperatures can be determined imperially using standard assays.

H. Pooling the Contents from Multiple Compartments

The pooling of contents from multiple compartments refers to the release of the labeled nucleic acid targets into a common medium with other similar labeled target molecules in such a way as they may interact with targets or labeling particles from disparate compartments. In some embodiments, this is a de-emulsification of water in oil droplets and can be achieved via the addition of a surfactant such as perfluorooctanol. In other embodiments, the compartments are a microwell array and the labeled target molecules are released by removal of a semi-permeable barrier.

Quenching of the free labeling primers may be accomplished prior to or during the pooling of contents from multiple compartments as described in Section II.G above. The pooling of compartments may also proceed through an intermediary step, where the contents of the compartment containing labeled nucleic acid target are exposed to the contents of another compartment or droplet prior to completion of the pooling. An example would be the mixing of a target nucleic acid containing compartments with an excess of compartments containing a quenching molecule prior to the addition of a surfactant. In this case, the probability of two compartments containing their respective free labeling primer fusing prior to exposure to the quenching molecule is limited during the pooling step. The compartments containing free labeling primer must first combine with compartments containing quenching particle due to proximity, leading to the quenching of the free labeling primers prior to labeling particles from disparate compartments observing another target nucleic acid during the pooling.

I. Next Generation Sequencing (NGS) Library Construction

After pooling the contents from multiple compartments into one continuous volume of aqueous solution, nucleic acid polymerase may be added to such aqueous solution to facilitate the extension of primer that is hybridized to the target nucleic acid, wherein the primer extension uses the target nucleic acid as the template.

In some embodiments, the compartments are water-in-oil droplets, wherein the pooling of contents from multiple compartments can be carried out by breaking the emulsion using methods described in Section II.H above.

In some embodiments, the nucleic acid polymerase is a RNA-dependent DNA polymerase. In some embodiments, the RNA-dependent DNA polymerase is a reverse transcriptase. In some embodiments, the reverse transcriptase is a native or engineered version of the reverse transcriptase from Moloney Murine Leukemia Virus (MMLT) or Avian Myeloblastosis Virus (AMV). In some embodiments, the reverse transcriptase is a SuperScript II, SuperScript III, or SuperScript IV.

In some embodiments, the nucleic acid polymerase is a DNA-dependent DNA polymerase.

In some embodiments, after pooling the contents from multiple compartments and before providing the nucleic acid polymerase, the pooled contents may be subject to DNA purification using routine methods. The DNA purification process may comprise using silica column, or using Solid Phase Reversible Immobilization (SPRI). SPRI may be carried out using Agencourt AMPure XP beads. The DNA purification process may help remove substances (such as fixation reversal agents, such as fixation reversal enzymes) that may interfere with the primer extension process catalyzed by the nucleic acid polymerase.

In some embodiments, it is desirable to provide the nucleic acid polymerase after pooling the contents from multiple compartments because (a) the compartments may contain substances (such as fixation reversal agents, such as fixation reversal enzymes) that may interfere with the primer extension process catalyzed by the nucleic acid polymerase, or (b) the compartments, before pooling, may have undergone treatment (such as heating to 60 degrees Celsius or above) that may compromise the quality of the target nucleic acid or the activity of the nucleic acid polymerase.

In some embodiments, providing the nucleic acid polymerase after pooling the contents of the compartments may create a problem: the primers and target nucleic acids that did not co-occupy a compartment may hybridize to each other and the primer can extend on the target nucleic acid. If the primer comprises a compartment barcode, this post-pooling hybridization and extension may lead to confused barcoding. In some embodiments, this problem is alleviated by providing quenching reagent in the compartments or during the pooling of the contents from the compartments using methods introduced in Paragraphs [0171] to [0188].

The product of the primer extension (e.g., the product of reverse transcription) can be further processed into NGS library using a variety of methods such as Smart-Seq, CEL-Seq, and their variations. Some of these methods are discussed in Svensson et al., Nat. Methods 14(4):381-387(2017).

### EXAMPLES

### Example 1

### Validating a Quenching Reagent

This example shows the procedure to validate that a reagent is a quenching reagent. As defined above, a quenching reagent is a reagent that (a) at optimal concentration, interferes with the interaction between target nucleic acid and primer such that the second-order rate constant for the interaction is reduced by at least 10-fold, but (b) at the above mentioned optimal concentration and under optimal experimental protocol, does not cause the dissociation of pre-formed complex between the target nucleic acid and the primer to a consequential extent, such that less than 50% of such pre-formed complex is dissociated during the experiment. This example also shows that when polyadenylated RNA is the target nucleic acid and dT₂₀ is a used as primer, then dA₅₀ can function as a quenching reagent.

In this example, the primer was a dT₂₀ oligo (SEQ ID NO: 1) which may mimic the primer released from a hydrogel bead and may freely associate with a target nucleic acid that is polyadenylated RNA. The association of the two nucleic acids then form a substrate for reverse transcription The putative quenching agent was a dA₅₀ (SEQ ID NO: 2) oligo with a sequence complimentary to that of the primer. Adding dA₅₀ to the RT reaction comprising free dT₂₀ may mimic the process of providing the quenching reagent during the pooling of contents from multiple compartments as described above.

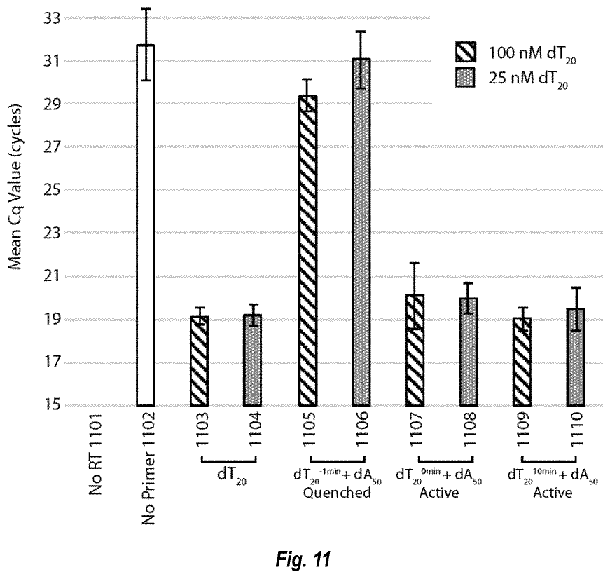

FIG. 10 shows the workflow and FIG. 11 shows the exemplary results of the workflow for a specific transcript of interest (GAPDH). Overall, the experiment showed that (a) if a high concentration of dA₅₀ is incubated with dT₂₀ before the dT₂₀ contacts polyadenylated RNA, the RT product is reduced by approximately 1,000-fold compared to the RT reaction in the absence of dA₅₀ (FIG. 11, compare columns 1103 and 1105); and (b) surprisingly, if dA₅₀ is added to the reaction after dT₂₀ contacts the polyadenylated RNA, the reduction in RT product is undetectable (FIG. 11, compare columns 1103 and 1109). These results show that the putative quench reagent dA₅₀ is a quenching reagent. The detail of the experiment is given below.

For all samples, the starting material was total RNA extract from leukocytes (Biochain) and an RT competent dT₂₀ primer (Integrated DNA Technologies (IDT)) (SEQ ID NO: 1). Reverse transcription (RT) was performed according to manufacturer's protocols (Invitrogen, Superscript IV) using 100 ng of RNA template. The product of the RT reaction was purified with AMPure XP beads (Agencourt). The purified products were quantitatively analyzed for the amount of cDNA produced by qPCR on a BioRad CFX96 Connect using NEB Luna Universal qPCR kits and IDT designed primers targeting GAPDH.

In FIG. 11, bar 1101 shows results from a control reaction assembled at room temperature containing 1× First Strand Buffer, 750 microMolar dNTPs, 100 nanograms Total RNA, and 150 nanoMolar dT₂₀ primer at a final volume of 13.5 microLiters. The sample was heated to 65° C. and then cooled to 25° C. (time point 1001 of FIG. 10). After incubation at 25° C. for 10 minutes (time point 1002 of FIG. 10), the sample was brought up to 20 microLiters in 1× First Strand Buffer and contained a final concentration of 5 microMolar DTT. The sample was then incubated for an additional 5 minutes at 25° C. prior to heating at 85° C. This reaction contained no reverse transcriptase enzyme. Therefore, no cDNA was synthesized and no amplification took place in the qPCR assay (giving no Cq value).

For bar 1102 of FIG. 11, the reaction condition was identical to the experiment described above, except (a) it contained no dT₂₀ primer and (b) 100 units of the reverse transcriptase enzyme were added along with the 1× First Strand buffer and DTT after the 10-minute incubation at 25° C. (time point 1002 of FIG. 10). This reaction yielded a high Cq value of 31.5 illustrating the low quantity of cDNA produced by the promiscuous extension activity of the reverse transcriptase in the absence of a primer. This Cq value represents the minimum amount of cDNA that can be expected to be produced for a sample that contains the SuperScript IV reverse transcriptase. Hence, this is the comparative data point for the degree of repression of RT activity.

The result from a positive control experiment is shown in bar 1103 of FIG. 11, and this experiment was identical to the one that led to bar 1101 of FIG. 11, except that 100 units of the RT enzyme was added at time point 1002 (see FIG. 10). Since the reaction that led to bar 1103 contains both reverse transcriptase and a primer, it was expected to produce the maximum amount of cDNA for these experimental conditions. Indeed, it yielded a low Cq value of 19.25. This value represented as the maximum achievable amount of cDNA of a completely unrestricted and unquenched reaction.

The reaction that led to bar 1105 of FIG. 11 was identical the one that led to bar 1102 of FIG. 11, except a mixture of 100 nanoMolar dT₂₀ (SEQ ID NO: 1) and 5 microMolar dA₅₀ (SEQ ID NO: 2) (i.e., the putative quenching molecule) was added after the 65° C. denaturation step (time point 1001 of FIG. 10). If dA₅₀ is a competent quenching reagent, the amount of cDNA produced would be close to the amount produced promiscuously by the RT in the absence of primer. Indeed, a Cq value resembling that of bar 1102 of FIG. 11 (the reaction that contained RT but no primer) was achieved, illustrating the ability of dA₅₀ to quench the dT₂₀ primer. And this Cq value is 10 cycles greater than that of bar 1103 of FIG. 11, suggesting a slowing of the interaction between the target nucleic acid and the primer by approximately 2¹⁰=˜1000 fold. This suggests that a target mRNA from a given compartment not previously exposed to a primer (dT₂₀ (SEQ ID NO: 1) in this embodiment) will not be labeled by free primer originating from a different compartment in the presence of quencher (dA₅₀ (SEQ ID NO: 2) in this embodiment).

The reaction that led to bar 1107 of FIG. 11 was identical to the reaction that lad to bar 1103 of FIG. 11, except dA₅₀ at a final concentration of 5 microMolar was added immediately after cooling to 25° C. from the denaturation at 65° C. (time point 1001 of FIG. 11). If dT₂₀ was associated with the target mRNA and the hybridization product was not disrupted by the dA₅₀ (i.e., the putative quenching reagent), cDNA should be synthesized to a high degree, resulting in a Cq value similar to that observed on bar 1103 of FIG. 11. This was indeed the case.

In another experiment, the addition of dA₅₀ was delayed and added with the RT after the 25° C. incubation (time point 1002 of FIG. 10). This led to the Cq value shown in bar 1109 of FIG. 11, which is undisguisable from the Cq value shown in bar 1103 of FIG. 11, the Cq value obtained in the positive control experiment. Since in this experiment the dT₂₀ and mRNA were given 10 min of time to associate, the results more clearly show that dA₅₀ does not cause the dissociation of the pre-formed complex between mRNA and the dT₂₀ primer.

The group of experiments that led to the results shown with bars 1104, 1106, 1108 and 1110 of FIG. 11 were the same as the group of experiments that led to the results shown with bars 1103, 1105, 1107 and 1109 of FIG. 11, except that the concentration of dT₂₀ primer used was different 25 nanoMolar. The data show that the observed phenomena are largely independent of the concentration of labeling primer present.

Collectively, this example demonstrates that when polyadenylated mRNA is the target nucleic acid and dT₂₀ is used as primer, then dA₅₀ is indeed a quenching reagent.

### Example 2

### scRNA-Seq Analysis of Nucleic Acid from FFPE Samples

This example provides methods to analyze single-cell transcriptomes in FFPE samples using nuclei as biological particles.

Current processes for qPCR analysis (Abrahamsen et al., J. Mol. Diagn. 5:34-41 (2003); Li et al., BMC Biotechnol. 8:10 (2008); Evers et al., J. Mol. Diagnostics 13:687-694 (2011)), flow cytometry (Hedley et al., J Histochem Cytochem 1333-1335 (1983); Jordanova et al., Am. J. Clin. Pathol. 120:327-334 (2003)), FISH (Paternoster et al., Am J Pathol 160: 1967-1972 (2002)), and population sequencing (Esteve-Codina et al., PLoS One 12: 1-18 (2017); Holley et al., PLoS One 7 (2012)) from archived samples, such as FFPE, rely on organic solvent to remove embedding wax, mechanical separation and enzymatic treatment to dissociate tissue, and a combination of heat treatment and enzymatic digestion for crosslink reversal in order to prepare samples for analysis. In the case of single cell encapsulation, preparation of single particles by, for example, xylene de-waxing and hyaluronidase and glycogenase treatment with passage through a Dounce homogenizer would result in single nuclei that are still crosslinked at a molecular level. However, the crosslinking is incompatible with polymerases (such as RT) and leads to reduced processivity. Reversal of the crosslinking with proteinase K and heat treatment would enable reverse transcription, but results in frail biological particles unable to be encapsulated into compartments on a relevant scale. (Paternoster, et al., Am J Pathol 160:1967-1972 (2002)). To solve this problem, crosslink reversal may be achieved or completed once the biological particles have been segregated into individual compartments.

Many current high throughput methods for compartmentalizing tens of thousands of biological particles rely on barcoded primers covalently attached to primer delivery particles (usually an immobilized phase, bead, or hydrogel). In one instance (Macosko et al., Cell 161:1202-1214 (2015)), the barcoded primers are attached to the bead and not mobilized during the experiment. In this case, target nucleic acids are hybridized to the immobilized barcoded primers on the primer delivery particles in the compartments. The primer delivery particles that are modified with barcoded primers and the target nucleic acids are then released from the compartment into a continuous volume of aqueous solution. A reverse transcriptase is then provided to extend the barcoded primer using the target nucleic acid as the template. In this method, no enzymatic nucleic acid copying process is done in the compartments, thus heating the compartments or providing protease in the compartments is not prohibited.

However, this method may be inefficient and undesirable due to the low labeling efficiency provided by an immobilized primer. (Macosko, et al., Cell 161:1202-1214 (2015)). Additionally, when compared to compartmentalized reverse transcription as described in the paragraph below, this method, at least in some implementations, may lead to an increased incidence of barcoded primers associating with target nucleic acids from a separate compartment resulting in confounded barcoding. Stoeckius et al., bioRxiv 113068 (2017).

In another method (Klein et al., Cell 161: 1187-1201 (2015)), the primers are mobilized (upon a stimulus such as UV light) from the beads and the primers can freely diffuse in the compartment that also includes the target nucleic acids of interest. This leads to an efficient labeling reaction that forms a competent RT substrate. Reverse transcription is performed in the compartment in the presence of a buffer comprising Mg⁺⁺ (which is a necessary cofactor of reverse transcriptase). Klein et al., Cell 161:1187-1201 (2015); Zilionis et al., Nat. Protoc. 12:44-73 (2016); and Jaitin et al., Science 343:776-779 (2014). This procedure is incompatible with heating due to RNA degradation in the presence of divalent metal ion, a step required for crosslink reversal. It is desired that the crosslink reversal is completed prior to reverse transcription. Additionally, if processing and reverse transcription were to take place after pooling the contents of the compartments, the free primers would be unrestricted in their ability to hybridize to and copy (via primer extension or reverse transcription) the target nucleic acids originating from a different compartment. This would confound the original intent of uniquely identifying the constituents of a biological particle in one given compartment.

This example provides methods for the processing of FFPE samples and analysis by RNA 3′ end sequencing in a favorable and desirable workflow allowing for the reversal of crosslinks while biological particles and target nucleic acid particles are still compartmentalized and efficient labeling with freely diffusing mobile primers. Isolation of nuclei from FFPE has been described. Holley et al., PLoS One 7 (2012). Prior to sorting, excess paraffin will be removed with a scalpel from either side of 40-60 μm scrolls to reduce accumulation of debris during the sorting process. The scroll will be collected into a micro-centrifuge tube then washed three times with 1 ml Xylene for 5 minutes to remove remaining paraffin. Each sample will be rehydrated in sequential ethanol washes (100% 5 minutes ×2, then 95%, 70%, 50% and 30% ethanol) and washed 2 times in 1 ml 1 mM EDTA pH 8.0.

The sample will be digested overnight (6-17 hours) in 1 ml of a freshly prepared enzymatic cocktail containing 50 units/ml of collagenase type 3, 80 units/ml of purified collagenase, and 100 units/ml of hyaluronidase in PBS pH 7.4/0.5 mM CaCl₂ buffer. Each enzyme will be rehydrated with PBS pH 7.4/0.5 mM CaCl₂ buffer, stored at −20° C., and thawed immediately prior to addition to make a cocktail mixture. Following overnight digestion, 500 microLiter NST will be added to each sample to facilitate pelleting. The sample will be centrifuged for 5 minutes at 3000×g, after which the pellet will be re-suspended in 750 microLiter of NST/10% fetal bovine serum and then passed through a 25 G needle or Dounce homogenizer 10-20 times. The sample will be filtered through a 35 micron mesh and collected into a 5 ml Polypropylene round bottom tube. The mesh will be rinsed with an additional 750 microLiter of NST/10% fetal bovine serum and placed on ice. The sample will then be counted on a hemocytometer, and then centrifuged for 5 minutes at 3000×g, after which the supernatant will be aspirated and the sample brought up to 100,000 nuclei per milliLiter of PBS (1 mM CaCl₂ and absent magnesium).

The sample will then be brought up to 16% v/v with Optiprep and placed on ice while the microfluidics device is prepared to encapsulate the individual nuclei. Compartmentalization of nuclei will be performed using the inDrop method as described. See, e.g., Klein et al., Cell 161:1187-1201 (2015); and Zilionis et al., Nat. Protoc. 12:44-73 (2016). The microfluidic device (80 mm deep) will be manufactured by soft lithography following standard protocols. During operation, nuclei suspension, reverse crosslinking/lysis mix, and collection tubes will be kept on ice. Flow rates will be 100 microLiter/hr for cell suspension, 100 microLiter/hr for reverse crosslinking/lysis mix, 10-20 microLiter/hr for barcoded hydrogel microspheres (BHMs), and 90 microLiter/hr for carrier oil to produce 4 nanoLiter drops. BHMs will serve as the primer delivery particles and will contain barcoded primers featuring (a) a photocleavable linker, (b) cell barcode, (c) UMI, and (d) dT₂₀ capable of hybridizing to mRNA poly A and serving as an RT primer. They will be prepared by washing, concentrated by centrifugation at 5000×g, and then loaded directly into tubing for injection into the device. The nuclei will be loaded directly into the syringe and maintained in suspension by the Optiprep. The carrier oil will be HFE-7500 fluorinated fluid (3M) with 0.75% (w/w) EA surfactant (RAN Biotechnologies). Reverse crosslinking/lysis mix will consist of 9 μL 10% (v/v) IGEPAL CA-630 (#18896 Sigma), 15 μL 1M TrisHCl [pH 8.0] (51238 Lonza), 15 μL RNAsecure (AM7005, Ambion), 50 μL proteinase K (800 U/ml; 40 units; P8107S NEB), 1 to 61 μL of 3 M potassium chloride, and sufficient nuclease-free water (AM9937 Ambion), to bring the total volume to 150 μL. After nuclei encapsulation, primers will be released by 8 min UV exposure (365 nm at 10 mW/cm², UVP B-100 lamp) while on ice. The emulsion will then be incubated at about 60° C. for 2-10 hr, then 10 to 60 min at about 90° C., then on ice. Since different samples may differ in aspects such as extent of crosslinking, the exact KCl amount in the crosslinking/lysis buffer, and the temperature and time for incubation will be optimized empirically to maximize the median length of the in vitro transcription (IVT) product (see the discussion below).

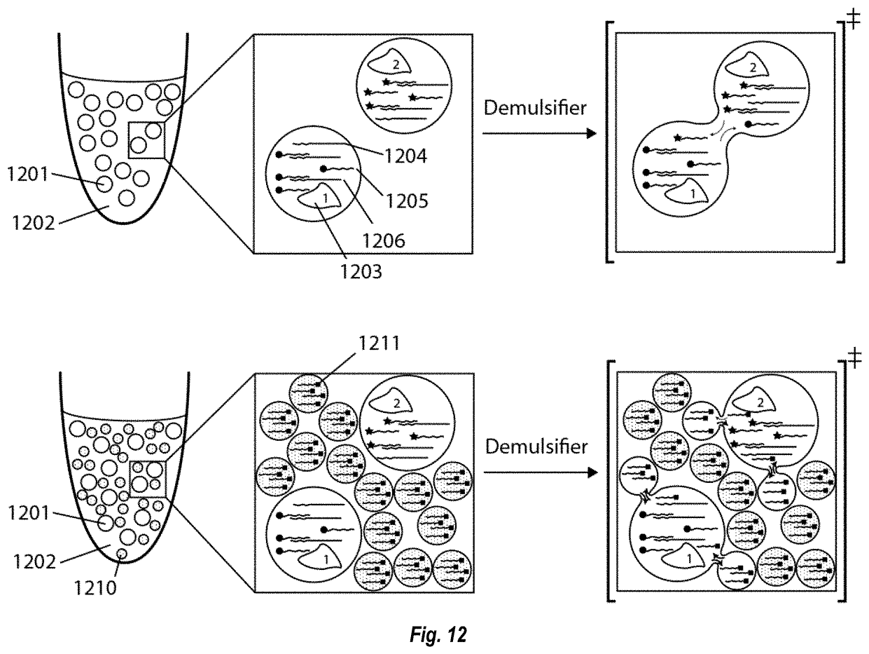

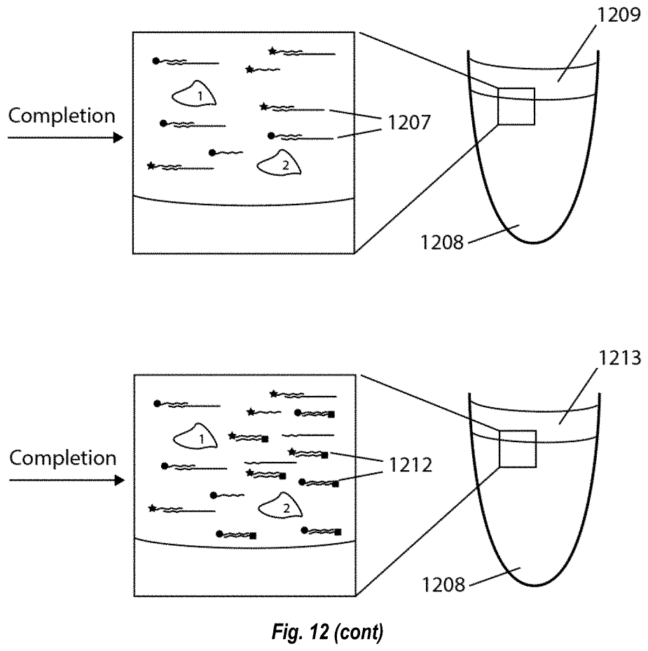

At this point, the crosslinking will have been reversed sufficiently to free the target nucleic acid (i.e., mRNA) and allow the barcoded primer to anneal. And hybridization between target nucleic acid and barcoded primer will have occurred. Next, the water-in-oil emulsion will be broken to pool the contents from different compartments. During this step, it is desirable that free primers (primers that are not hybridized with target nucleic acid) are not able to interact with target nucleic acids from a different compartment. FIG. 12 shows the most common means of demulsification, achieved through the addition of a surfactant (perfluorooctanol in this instance). See, e.g., Zolfaghari et al., Sep. Purif. Technol. 170:377-407 (2016). In FIG. 12, two different cell barcode sequences are represented by filled circles and stars. Aqueous droplets (FIG. 12, 1201) suspended in oil (FIG. 12, 1202) contain a heterogeneous mixture consisting of remnants of the biological particle (FIG. 12, 1203), the target mRNA (FIG. 12, 1204), the mobilized free primer (FIG. 12, 1205), and primer-target nucleic acid complex (FIG. 12, 1206). The mechanism of demulsification progresses through a transition state (FIG. 12, ‡) which involves the fusion of individual compartments when a demulsifier is added, leading to the mixing of the internal constituents and thus the biological particles of one compartment are exposed at high concentration to the free primers of an adjacent compartment. This may allow barcoded primers from one compartment to label target mRNAs from another compartment, resulting in undesirable primer-target nucleic acid complexes (FIG. 12, 1207) in the final aqueous phase (FIG. 12, 1209) sitting atop the carrier oil (FIG. 12, 1208) from the microfluidics chip. In order to combat this, quenching droplets (FIG. 12, 1210) designed to fuse with the compartments containing mobilized primers and target nucleic acids will be generated as water-in-oil emulsions or droplets containing a 1 mM solution of dA₅₀ as a quenching reagent (FIG. 12, 1211), as described and validated in Example 1. These quenching droplets will then be added at a 100:1 ratio into the compartments containing the primer and target nucleic acids, mixed gently, and demulsified by adding 0.2×20% (v/v) perfluorooctanol, 80% (v/v) HFE-7500 and brief centrifugation. This will result in negligible amounts of compartment cross contamination due to the robust hybridization of the quencher dA₅₀, forming waste product (FIG. 12, 1212) which can be easily removed for downstream processing.

The aqueous phase of the broken droplets (FIG. 12, 1213) will then be placed in a 30 k MWCO filter (UFC503008 Millipore) and centrifuged at 14,000 rcf to remove the quenching reagent (FIG. 12, 1211) and waste product (FIG. 12, 1212). The sample will then be concentrated by washing with two volumes of ice cold PBS to a volume of ˜50 μL. The remaining primer-target nucleic acid complexes present on top of the filtered solution will then be added to the reverse transcription reaction described in the next paragraph. Alternatively, the quenching reagent (FIG. 12, 1211) and waste product (FIG. 12, 1212) may be removed using standard SPRI protocol. They may also be left in the sample and the method may still generate acceptable results.

The product from the previous step will be added to a reverse transcription reaction containing 25 μL, 5× First-Strand buffer (18080-044 Life Technologies), 6 μL 25 mM dNTPs (Enzymatics N2050L), 10 μL 0.1M DTT (#18080-044, Life Technologies), 15 μL 1M TrisHCl [pH 8.0] (51238 Lonza), 10 μL Murine RNase inhibitor (M0314, NEB), 15 μL SuperScript III RT enzyme (200 U/μL, #18080-044, Life Technologies), and volume adjusted to 150 μL with nuclease-free water (AM9937 Ambion). The mixture will then be held at 50° C. for 2 hours, then 70° C. for 15 minutes, and then on ice. The sample can be purified with AMPure XP beads (A63880 Beckman) or proceed directly to library preparation.

The resulting solution will contain individual compartment barcoded cDNA from FFPE samples. Standard workflows will be implemented from this point forward for second strand synthesis using NEBnext UltraII (E7771 NEB) and in vitro transcription (IVT) using PrimeScript (6111A Clontech) and ensuing steps described in Zilionis et al., (2017) Nat Protoc 12: 44. Alternatively library preparation may also be carried out using the Smart-seq2 (Picelli et al., Nat. Protoc. 9:171-181 (2014)). or CEL-Seq2 methods (Hashimshony et al., Genome Biol. 17:77 (2016)). DNA libraries resulting from these protocols will be sequenced on, for example a NextSeq500 or HiSeq2500 Illumina sequencer. As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as “at least” and “about” precede a list of numerical values or ranges, the terms modify all of the values or ranges provided in the list. In some instances, the term “about” may include numerical values that are rounded to the nearest significant figure.

### BIBLIOGRAPHY

1. Klein, A. M. et al. Droplet barcoding for single-cell transcriptomics applied to embryonic stem cells. Cell 161, 1187-1201 (2015).
2. Krishnaswami, S R. et al. Using single nuclei for RNA-seq to capture the transcriptome of postmortem neurons. Nat. Protoc. 11, 499-524 (2016).
3. Zheng, G. X. Y. et al. Massively parallel digital transcriptional profiling of single cells. Nat. Commun. 8, 14049 (2017).
4. Gierahn, T. M. et al. Seq-Well: portable, low-cost RNA sequencing of single cells at high throughput. Nat. Methods 14:395-398 (2017). doi:10.1038/nmeth.4179
5. Macosko, E. Z. et al. Highly parallel genome-wide expression profiling of individual cells using nanoliter droplets. Cell 161, 1202-1214 (2015).
6. Svensson, V. et al. Power analysis of single-cell RNA-sequencing experiments. Nat. Methods 14(4):381-387 (2017). doi:10.1038/nmeth.4220
7. Zhu, P. & Wang, L. Passive and active droplet generation with microfluidics: a review. Lab Chip 17, 34-75 (2017).
8. Gierahn, T. M. et al. Seq-Well: portable, low-cost RNA sequencing of single cells at high throughput. Nat. Methods 14:395-398 (2017). doi:10.1038/nmeth.4179
9. Zilionis, R. et al. Single-cell barcoding and sequencing using droplet microfluidics. Nat. Protoc. 12, 44-73 (2016).
10. Hindson, B. et al. Polynucleotide barcode generation. (2016).
11. Wegner, S. V., Sentürk, O. I. & Spatz, J. P. Photocleavable linker for the patterning of bioactive molecules. Sci. Rep. 5, 18309 (2016).
12. Karmakar, S. et al. Organocatalytic removal of formaldehyde adducts from RNA and DNA bases. Nat. Chem. 7, 752-758 (2015). doi:10.1038/nchem.2307
13. Ellefson, J. W. et al. Synthetic evolutionary origin of a proofreading reverse transcriptase. Science (80-.). 352, 1590-1593 (2016).
14. Simard, C., Lemieux, R. & Côté, S. Urea substitutes toxic formamide as destabilizing agent in nucleic acid hybridizations with RNA probes. Electrophoresis 22, 2679-2683 (2001).
15. Moore, D. & Dowhan, D. in Current Protocols in Molecular Biology 22, 447-450 (John Wiley & Sons, Inc., 2002).
16. Spencer, S. et al. epicPCR (Emulsion, Paired Isolation, and Concatenation PCR). Protoc. Exch. (2015). doi:10.1038/nbt0798-652
17. Villani, A.-C. et al. Single-cell RNA-seq reveals new types of human blood dendritic cells, monocytes, and progenitors. Science 356, 6335 (2017).
18. Tawfik, D. S. & Griffiths, A. D. Man-made cell-like compartments for molecular evolution. Nat. Biotechnol. 16, 652-656 (1998).
19. Kim, Y., Phillips, J. a, Liu, H., Kang, H. & Tan, W. Using photons to manipulate enzyme inhibition by an azobenzene-modified nucleic acid probe. Proc. Natl. Acad. Sci. 106, 6489-6494 (2009).
20. Connelly, C. M., Uprety, R., Hemphill, J. & Deiters, A. Spatiotemporal control of microRNA function using light-activated antagomirs. Mol. Biossyst. 8, 2987 (2012).
21. Abrahamsen, H. N., Steiniche, T., Nexo, E., Hamilton-Dutoit, S. J. & Sorensen, B. S. Towards quantitative mRNA analysis in paraffin-embedded tissues using real-time reverse transcriptase-polymerase chain reaction: a methodological study on lymph nodes from melanoma patients. J. Mol. Diagn. 5, 34-41 (2003).
22. Li, J. et al. Improved RNA quality and TaqMan® Pre-amplification method (PreAmp) to enhance expression analysis from formalin fixed paraffin embedded (FFPE) materials. BMC Biotechnol. 8, 10 (2008).
23. Evers, D. L., He, J., Kim, Y. H., Mason, J. T. & O'Leary, T. J. Paraffin embedding contributes to RNA aggregation, reduced RNA yield, and low RNA quality. J. Mol. Diagnostics 13, 687-694 (2011).
24. Hedley, D. W., Friedlander, M. L., Taylor, I. W., Rugg, C. A. & Musgrove, E. A. Method for analysis of cellular DNA content of paraffin-embedded pathological material using flow cytometry. J Histochem Cytochem 1333-1335 (1983).
25. Jordanova, E. S. et al. Flow Cytometric Sorting of Paraffin-Embedded Tumor Tissues Considerably Improves Molecular Genetic Analysis. Am. J. Clin. Pathol. 120, 327-334 (2003).
26. Paternoster, S. F. et al. A new method to extract nuclei from paraffin-embedded tissue to study lymphomas using interphase fluorescence in situ hybridization. Am J Pathol 160, 1967-1972 (2002).
27. Esteve-Codina, A. et al. A comparison of RNA-Seq results from paired formalin-fixed paraffin-embedded and fresh-frozen glioblastoma tissue samples. PLoS One 12, 1-18 (2017).
28. Holley, T. et al. Deep Clonal Profiling of Formalin Fixed Paraffin Embedded Clinical Samples. PLoS One 7, (2012).
29. Stoeckius, M. et al. Large-scale simultaneous measurement of epitopes and transcriptomes in single cells. bioRxiv 113068 (2017). doi:10.1101/113068
30. Jaitin, D. A. et al. Massively Parallel Single-Cell RNA-Seq for Marker-Free Decomposition of Tissues into Cell Types. Science 343, 776-779 (2014).
31. Zolfaghari, R., Fakhru?l-Razi, A., Abdullah, L. C., Elnashaie, S. S. E. H. & Pendashteh, A. Demulsification techniques of water-in-oil and oil-in-water emulsions in petroleum industry. Sep. Purif. Technol. 170, 377-407 (2016).
32. Picelli, S. et al. Full-length RNA-seq from single cells using Smart-seq2. Nat. Protoc. 9, 171-181 (2014).
33. Hashimshony, T. et al. CEL-Seq2: sensitive highly-multiplexed single-cell RNA-Seq. Genome Biol. 17, 77 (2016).

What is claimed is:

1. A method of labeling at least one target nucleic acid molecule from a biological particle with a barcoded primer, comprising:
a. providing a pool of at least about 100 biological particles, wherein the biological particles comprise at least one target nucleic acid molecule;
b. partitioning the pool of biological particles into compartments, wherein at least some of the compartments contain a primer delivery particle, wherein the primer delivery particle contains barcoded primers comprising at least 5 consecutive nucleotides that are complementary to at least a portion of the at least one target nucleic acid of the biological particle; and wherein the at least one barcoded primer binds to at least one target nucleic acid; and
c. inactivating barcoded primers that are not bound to a target nucleic acid.

2. The method of claim 1, further comprising mobilizing the barcoded primers from the primer delivery particle.

3. The method of any one of claims 1-2, wherein at least 50% of the compartments contain no more than one biological particle.

4. The method of any one of claims 1-2, wherein at least 60%, 70%, 75%, 80%, 85%, 90%, 95%, 99%, or 100%of the compartments contain no more than one biological particle.

5. The method of any one of claims 1-4, wherein at least 50% of the compartments contain no more than one primer delivery particle.

6. The method of any one of claims 1-4, wherein at least 60%, 70%, 75%, 80%, 85%, 90%, 95%, 99%, or 100% of the compartments contain no more than one primer delivery particle.

7. The method of any one of claims 1-6, further comprising heating the compartments containing the biological particles to a temperature of about 60 degrees Celsius for at least about 10 minutes.

8. The method of any one of claims 1-7, further comprising providing one or more proteases, one or more fixation reversal agents, or any combinations thereof in the compartment.

9. The method of claim 8, wherein one or more fixation reversal agents comprise at least one fixation reversal catalyst.

10. The method of claim 8, wherein one or more fixation reversal agents comprise at least one fixation reversal enzyme.

11. The method of any one of claims 1-10, further comprising fixing the biological particles with one or more fixatives prior to partitioning the pool of biological particles into compartments.

12. The method of any one of claims 1-11, further comprising inactivating the barcoded primers that are not bound to any target nucleic acid by photo-cleaving at least one inhibitor oligonucleotide whose sequence is partially or entirely complementary to the barcoded primer.

13. The method of any one of claims 1-12, further comprising inactivating the barcoded primers that are not bound to any target nucleic acid by
a. providing a quencher that can bind to either the barcoded primers or the target nucleic acid under a lower temperature condition and
b. incubating the compartments at a first temperature for at least 5 minutes and then incubating the compartments at a second temperature for at least 30 seconds, wherein the second temperature is lower than the first temperature by at least 5 degrees Celsius;
c. and allowing the quencher to inactivate the barcoded primers at the lower temperature condition.

14. The method of any of claims 1-12, further comprising inactivating the barcoded primers that are not bound to any target nucleic acid by
a. providing a quencher reagent that can bind to either the barcoded primers or the target nucleic acid and can be inactivated by a temperature-sensitive secondary quencher at a higher temperature condition;
b. incubating the compartments at a first temperature for at least 5 minutes and then incubating the compartments at a second temperature for at least 30 seconds, wherein the second temperature is higher than the first temperature by at least 5 degrees Celsius; and
c. allowing the quencher to inactivate the barcoded primers at the higher temperature condition.

15. The method of any of claims 1-12, further comprising inactivating the barcoded primers that are not bound to any target nucleic acid with at least one inhibitor oligonucleotide whose sequence is partially or entirely complementary to the barcoded primers.

16. The method of any of claims 1-12, further comprising inactivating the barcoded primers that are not bound to any target nucleic acid with at least one interfering reagent.

17. The method of claim 16, wherein the at least one interfering reagent comprises nucleic acid precipitants, dimethyl sulfoxide (DMSO), betaines, polyamines, urea, formamide, metal ion chelators, and combinations thereof.

18. The method of claim 8 or 13-17, wherein the inhibitor oligonucleotide or interfering reagent is in a water-in-oil emulsion.

19. A method of labeling at least one target nucleic acid molecule from a biological particle with a barcoded primer, comprising:
a. providing a pool of at least 100 biological particles, wherein the biological particles comprise at least one target nucleic acid;
b. partitioning the pool of biological particles into compartments wherein at least some of compartments contain a primer delivery particle, wherein the primer delivery particle contains barcoded primers comprising at least 5 consecutive nucleotides that are complementary to at least a portion of at least one target nucleic acid of the biological particle; and wherein the at least one barcoded primer binds to at least one target nucleic acid; and
c. mobilizing the barcoded primers from the primer delivery particles before and/or after the binding of at least one barcoded primer to at least one target nucleic acid; and
d. heating the compartments accommodating the biological particles at a temperature of at least 80 degrees Celsius for at least 10 min

20. The method of claim 19, wherein the compartments further comprise at least one protease, at least one fixation reversal agent, or both.

21. The method of any of the claims 19-20, further comprising fixing the biological particles with one or more fixatives prior to partitioning the pool of biological particles into compartments.

22. A method of labeling at least one target nucleic acid molecule from a biological particle with a barcoded primer, comprising:
a. providing a pool of at least 100 biological particles, wherein the biological particles comprise at least one target nucleic acid;
b. partitioning the pool of biological particles into compartments, wherein at least some of the compartments contain a primer delivery particle, wherein the primer delivery particle contains barcoded primers comprising at least 5 consecutive nucleotides that are complementary to at least a portion of at least one target nucleic acid of the biological particle; and wherein the at least one barcoded primer binds to at least one target nucleic acid;
c. mobilizing the barcoded primers from the primer delivery particle before and/or after the binding of at least one barcoded primer to at least one target nucleic acid; and
d. providing a fixation reversal agent in the compartments.

23. The method of claim 22, further comprising fixing the biological particles with one or more fixatives prior to partitioning the pool of biological particles into compartments.

24. A method of labeling at least one target nucleic acid molecule from a biological particle with a barcoded primer, comprising:
a. providing a pool of at least 100 biological particles, wherein the biological particles comprise at least one target nucleic acid;
b. partitioning the pool of biological particles into compartments wherein at least some of the compartments contain a primer delivery particle, wherein the primer delivery particle contains barcoded primers comprising at least 5 consecutive nucleotides that are complementary to at least a portion of at least one target nucleic acid of the biological particle, and wherein the at least one barcoded primer binds to at least one target nucleic acid; and
c. (i) mobilizing the barcoded primers from the primer delivery particle in the compartments before and/or after the binding of at least one barcoded primer to at least one target nucleic acid, (ii) after mobilizing the barcoded primers, pooling the contents of the compartments into an aqueous solution, and (iii) after pooling the contents, contacting the pooled contents in the aqueous solution with one or more nucleic acid polymerase.

25. The method of claim 24, wherein the nucleic acid polymerase is a RNA-dependent DNA polymerase.

26. The method of claim 25, wherein the RNA-dependent DNA polymerase is a reverse transcriptase.

27. The method of claim 24, wherein the nucleic acid polymerase is a DNA-dependent DNA polymerase.

28. The method of any of claims 2-27, wherein the barcoded primers are mobilized from the primer delivery particle by UV illumination, one or more reducing agents that reduce disulfide bonds, one or more enzymes that break any covalent bond between the barcoded primer and the primer delivery particle, or one or more enzymes that degrade the primer delivery particle.

29. The methods of any one of claims 1-28, wherein the median volume of the aqueous content in the compartments is 1 microLiter or less.

30. The method of any one of claims 1-29, wherein the compartments are droplets.

31. The method of any one of claims 1-30, wherein the biological particles are cells.

32. The method of claim 31, wherein at least some of the cells are prokaryotic cells.

33. The method of claim 31-32, wherein at least some of the cells are eukaryotic cells.

34. The method of claim 31-33, wherein at least some of the cells are engineered with DNA, RNA or viral vectors that encode one or more biological agents that cause RNA-mediated gene knockdown, genome editing, transcriptional alteration, or epigenetic alteration.

35. The method of claim 34, wherein the one or more biological agents comprise one or more of siRNA, shRNA, miRNA, zinc finger domains, transcription activator-like effector (TALE), Cas9, RNA with CRISPR origin.

36. The method of any one of claims 1-35, wherein the target nucleic acid is RNA.

37. The method of any of claims 1-35, wherein the target nucleic acid is DNA.

38. The method of any one of claims 1-37, wherein the target nucleic acid is at least part of an engineered molecule that is used to engineer or probe the biological particle.

39. The method of any one of claims 1-38, wherein the pool of biological particles is partitioned into at least 100 compartments.

40. The method of any one of claims 1-39, wherein at least 1% of the compartments contain a primer delivery particle.

41. The method of any one of claims 1-40, wherein at least 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more primer delivery particles are partitioned into compartments.

42. The method of any one of claims 1-41, wherein at least 2, 5, 10, 50, 100, 250, 500, 750, 1000, 1500, 2000, 2500, 5000, 7500, or 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, 20000000, or more biological particles are partitioned into compartments.

43. The method of any one of claims 1-42, wherein at least some of the barcoded primers that are not bound to a target nucleic acid are inactivated in the compartments before pooling of the contents of the compartments into an aqueous solution.

44. The method of any one of claims 1-43, wherein at least some of the barcoded primers that are not bound to a target nucleic acid are inactivated in the compartments during pooling of the contents of the compartments into an aqueous solution.

45. The method of any one of claims 1-44, wherein at least some of the barcoded primers that are not bound to a target nucleic acid are inactivated in the compartments after pooling of the contents of the compartments into an aqueous solution.
